SEQUENCE LISTING (30 sequences)

SEQ ID NO: 1 - protein, 32 aa, Synthetic construct
AGDRADGQPAGDRADGQPAGDRADGQPAGDRC

SEQ ID NO: 2 - protein, 32 aa, Synthetic construct
ANGAGNQPGANGAGNQPGANGAGNQPGANGAC

SEQ ID NO: 3 - protein, 32 aa, Synthetic construct
AQPDGPAQNAQPDGPAQNAQPDGPAQNAQPDC

SEQ ID NO: 4 - protein, 31 aa, Synthetic construct
CGDRADGQPAGDRADGQPAGDRADGQPAGDR

SEQ ID NO: 5 - protein, 31 aa, Synthetic construct
CANGAGNQPGANGAGNQPGANGAGNQPGANG

SEQ ID NO: 6 - protein, 19 aa, Synthetic construct
CAGPNSRDTQAGPNSRDTQ

SEQ ID NO: 7 - protein, 342 aa, Synthetic construct
GLNDIFEAQKIEWHEGSGSGTHCGHNVDLSKAINLNGVNFNNVDASSLGAAHVGQSASRGRGLGENPDDEEGDAKKKKDGKKAEPKNPRENKLKQPAGDRADGQPAGDRADGQPAGDRADGQPAGDRAAGQPAGDRADGQPAGDRAAGQPAGDRADGQPAGDRAAGQPAGDRADGQPAGDRAAGQPAGDRAAGQPAGDRADGQPAGDRAAGQPAGDRADGQPAGDRAAGQPAGDRAAGQPAGNGAGGQAAGGNAGGNAGGNAGGNAGGQGQNNEGANAPNEKSVKEYLDKVRATVGTEWTPCSVTCGVGVRVRRRVNAANKKPEDLTLNDLETDVCTMDKCA

SEQ ID NO: 8 - protein, 373 aa, Synthetic construct
GNDIFEAQKIEWHEGSGSGTHCGHNVDLSKAINLNGVGFNNVDASSLGAAHVGQSASRGRGLGENPDDEEGDAKKKKDGKKAEPKNPRENKLKQPEDGAGNQPGANGAGNQPGANGAGNQPGANGAGDQPGANGAGNQPGANGAGDQPGANGAGNQPGANGAGNQPGANGAGNQPGANGADDQPGANGAGNQPGANGAGNQPGANGAGNQPGANGAGDQPGANGAGNQPGANGAGDQPGANGAGNQPGANGAGNQPGANGAGNQPGANGAGNQPGANGAGGQAAGGNAANKKAGDAGAGQGQNNEGANATNEKSVKEYLDKVRATVGTEWTPCSVTCGVGVRVRRRVNAANKKPEDLTLNDLETDVCTMDKCA

SEQ ID NO: 9 - protein, 358 aa, Synthetic construct
GLNDIFEAQKIEWHEGSGSGPTHCGHNVDLSKAINLNGVNFNNVDASSLGAAHVGQSASRGRGLGENPDDEEGDAKKKKDGKKAEPKNPRENKLKQPGDRADGQPAGDRADGQPAGDRADGQPAGDRAAGQPAGDRADGQPAGDRADGQPAGDRADGQPAGDRADGQPAGDRAAGQPAGDRAAGQPAGDRADGQPAGDRAAGQPAGDRADGQPAGDRAAGQPAGDRADGQPAGDRAAGQPAGDRAAGQPAGDRAAGQPAGDRAAGQPAGNGAGGQAAGGNAGGGQGQNNEGANAPNEKSVKEYLDKVRATVGTEWTPCSVTCGVGVRVRRRVNAANKKPEDLTLNDLETDVCTMDKCA

SEQ ID NO: 10 - protein, 96 aa, Synthetic construct
GLNDIFEAQKIEWHEGSGSGTHCGHNVDLSKAINLNGVNFNNVDASSLGAAHVGQSASRGRGLGENPDDEEGDAKKKKDGKKAEPKNPRENKLKQP

SEQ ID NO: 11 - protein, 96 aa, Synthetic construct
GLNDIFEAQKIEWHEGSGSGGGQGQNNEGANAPNEKSVKEYLDKVRATVGTEWTPCSVTCGVGVRVRRRVNAANKKPEDLTLNDLETDVCTMDKCA

SEQ ID NO: 12 - protein, 177 aa, Synthetic construct
GLNDIFEAQKIEWHEGSGSGTHCGHNVDLSKAINLNGVNFNNVDASSLGAAHVGQSASRGRGLGENPDDEEGDAKKKKDGKKAEPKNPRENKLKQPGSGSGGGQGQNNEGANAPNEKSVKEYLDKVRATVGTEWTPCSVTCGVGVRVRRRVNAANKKPEDLTLNDLETDVCTMDKCA

SEQ ID NO: 13 - protein, 322 aa, unidentified
  source 1..322: note = Plasmodium vivax CSP VK210
THCGHNVDLSKAINLNGVNFNNVDASSLGAAHVGQSASRGRGLGENPDDEEGDAKKKKDGKKAEPKNPRENKLKQPAGDRADGQPAGDRADGQPAGDRADGQPAGDRAAGQPAGDRADGQPAGDRAAGQPAGDRADGQPAGDRAAGQPAGDRADGQPAGDRAAGQPAGDRAAGQPAGDRADGQPAGDRAAGQPAGDRADGQPAGDRAAGQPAGDRAAGQPAGNGAGGQAAGGNAGGNAGGNAGGNAGGQGQNNEGANAPNEKSVKEYLDKVRATVGTEWTPCSVTCGVGVRVRRRVNAANKKPEDLTLNDLETDVCTMDKCA

SEQ ID NO: 14 - protein, 337 aa, unidentified
  source 1..337: note = Plasmodium vivax CSP Belem
THCGHNVDLSKAINLNGVNFNNVDASSLGAAHVGQSASRGRGLGENPDDEEGDAKKKKDGKKAEPKNPRENKLKQPGDRADGQPAGDRADGQPAGDRADGQPAGDRAAGQPAGDRADGQPAGDRADGQPAGDRADGQPAGDRADGQPAGDRAAGQPAGDRAAGQPAGDRADGQPAGDRAAGQPAGDRADGQPAGDRAAGQPAGDRADGQPAGDRAAGQPAGDRAAGQPAGDRAAGQPAGDRAAGQPAGNGAGGQAAGGNAGGGQGQNNEGANAPNEKSVKEYLDKVRATVGTEWTPCSVTCGVGVRVRRRVNAANKKPEDLTLNDLETDVCTMDKCA

SEQ ID NO: 15 - protein, 354 aa, unidentified
  source 1..354: note = Plasmodium vivax CSP VK247
THCGHNVDLSKAINLNGVGFNNVDASSLGAAHVGQSASRGRGLGENPDDEEGDAKKKKDGKKAEPKNPRENKLKQPEDGAGNQPGANGAGNQPGANGAGNQPGANGAGDQPGANGAGNQPGANGAGDQPGANGAGNQPGANGAGNQPGANGAGNQPGANGAADQPGANGAGNQPGANGAGNQPGANGAGNQPGANGAGDQPGANGAGNQPGANGAGDQPGANGAGNQPGANGAGNQPGANGAGNQPGANGAGNQPGANGAGGQAAGGNAANKKAGDAGAGQGQNNEGANATNEKSVKEYLDKVRATVGTEWTPCSVTCGVGVRVRRRVNAANKKPEDLTLNDLETDVCTMDKCA

SEQ ID NO: 16 - DNA, 44 bp, synthetic construct
  source 1..44: mol_type = other DNA
aagcttgggccctcgagcaagcatatacataaaagggaatatgg

SEQ ID NO: 17 - DNA, 44 bp, Synthetic construct
  source 1..44: mol_type = other DNA
gtcgactttaaatatatgcgtgtatatatagattttgttttttg

SEQ ID NO: 18 - DNA, 39 bp, synthetic construct
  source 1..39: mol_type = other DNA
cgtcgacataaacattacgcatgattataaatatttata

SEQ ID NO: 19 - DNA, 43 bp, synthetic construct
  source 1..43: mol_type = other DNA
gaattctcgagaattaaccaatgctgtatactattataatgtc

SEQ ID NO: 20 - DNA, 22 bp, synthetic construct
  source 1..22: mol_type = other DNA
ccaacacactgtggtcacaacg

SEQ ID NO: 21 - DNA, 29 bp, synthetic construct
  source 1..29: mol_type = other DNA
caacgttaaatataccggcacacttatcc

SEQ ID NO: 22 - DNA, 34 bp, synthetic construct
  source 1..34: mol_type = other DNA
caattcctctttcaatctgtacatatttattctc

SEQ ID NO: 23 - DNA, 25 bp, synthetic construct
  source 1..25: mol_type = other DNA
tctacgttgtgaccacagtgtgttg

SEQ ID NO: 24 - DNA, 31 bp, synthetic construct
  source 1..31: mol_type = other DNA
cattgaatgaccttgagactgatgtttgtac

SEQ ID NO: 25 - DNA, 37 bp, synthetic construct
  source 1..37: mol_type = other DNA
gtatttttattgaaaaagacacaaaatagctagtttg

SEQ ID NO: 26 - DNA, 27 bp, synthetic construct
  source 1..27: mol_type = other DNA
ggtattccacgctgtgttgtcggtgag

SEQ ID NO: 27 - DNA, 32 bp, synthetic construct
  source 1..32: mol_type = other DNA
gttcataattttagctatttacatgcatgtgc

SEQ ID NO: 28 - DNA, 30 bp, synthetic construct
  source 1..30: mol_type = other DNA
atgaagaagtgtaccattttagttgtagcg

SEQ ID NO: 29 - DNA, 32 bp, synthetic construct
  source 1..32: mol_type = other DNA
gtatcaatatcttctaaggtcaaatcttctgc

SEQ ID NO: 30 - DNA, 1129 bp, synthetic construct
  source 1..1129: mol_type = other DNA
tatacacgcatatatttaaaatgaaaaactttattttattagcagtatcttctatcttattagttgacttatttccaacacactgtggtcacaacgtagacttatctaaagcgataaatttaaatggtgttaactttaataatgttgacgcatcatcattaggagcagcccacgttggtaaagcgcatcacgaggtcgtggattaggtgaaaatccagacgacgaagagggtgacgcaaaaaaaaaaaaggacggaaaaaaagcagaaccaaaaaatccacgtgaaaataagttaaaacaaccagcaggtgaccgagcagacggacaaccagcaggtgaccgagcagacggacagccagcaggtgacagagcagacggacagccagcaggtgaccgggcagccggacaaccagcaggtgacagggcagacggacagccagcaggtgacagggcagcgggacaaccagcaggtgacagagcagacggacagccagcaggagaccgagcagccggacaaccagcaggtgatcgagcagacggacagccagcaggagacagggcagcgggacaaccagcaggtgatagagcagcgggacaaccagcaggtgatagagcagatggacagccagcaggtgatagggcagctggacaaccagcaggtgatagagcagatggacaaccagcaggagatagagcagctggacagccagcaggagatagagcagctggacagccagcaggaaatggtgcaggtggacaggcagcaggaggaaacgcaggaggaaacgcaggaggaaacgcaggaggaaacgcaggaggacagggacaaaataatgaaggtgcgaatgccccaaatgaaaaatctgtaaaagaatacctagataaagttagagccaccgttggtaccgaatggacaccatgtagtgtaacctgtggagtaggtgtacgagtacgacggcgagttaatgcagcgaacaaaaaaccagaggatcttacattgaatgaccttgagactgatgtttgtacaatggataagtgtgccggtatatttaacgttgtgagtaattcattaggtctagtcatattgttagttctagcattattcaattaaataaacattacgcatgat

(19) United States
(12) Patent Application Publication
(10) Pub. No.: US 2025/0339505 A1
(43) Pub. Date: Nov. 6, 2025

(54) IMMUNOGENICITY OF PLASMODIUM VIVAX CIRCUMSPOROZOITE PROTEIN NANOPARTICLE VACCINES

(71) Applicant: UNIVERSITY OF SOUTH FLORIDA, Tampa, FL (US)

(72) Inventors: Francis NTUMNGIA, Tampa, FL (US); John ADAMS, Tampa, FL (US); Samantha BARNES, Tampa, FL (US)

(21) Appl. No.: 19/067,592

(22) Filed: Feb. 28, 2025

Related U.S. Application Data
(60) Provisional application No. 63/559,945, filed on Mar. 1, 2024.
(60) Provisional application No. 63/719,718, filed on Nov. 13, 2024.

(51) Int. Cl. A61K 39/015; A61K 39/00; A61P 37/04; C07K 14/445

(52) U.S. Cl. CPC A61K 39/015; A61P 37/04; C07K 14/445; A61K 2039/6093

(57) ABSTRACT

Provided herein are antibodies and pharmaceutical compositions for preventing malaria disease, and methods of their use.

### CROSS REFERENCE TO RELATED APPLICATIONS

This application claims the benefit of and priority to U.S. Provisional Application Ser. No. 63/559,945, filed Mar. 1, 2024 and U.S. Provisional Application Ser. No. 63/719,718, filed Nov. 13, 2024, the entireties of which are incorporated herein by reference.

### STATEMENT REGARDING FEDERALLY SPONSORED RESEARCH OR DEVELOPMENT

This invention was made with government support under grant number U01AI155361 awarded by the National Institutes of Health. The government has certain rights in the invention.

### REFERENCE TO A SEQUENCE LISTING

The content of the electronic sequence listing (File name: 173738_02877.xml; Size 30.1 k bytes; Date of Creation: Feb. 28, 2025) is herein incorporated by reference in its entirety.

### BACKGROUND

The field of the disclosure relates to nanoparticle compositions and methods for inducing an immune response against the circumsporozoite protein (CSP) of Plasmodium vivax to protect against malaria infection. In particular, the field of the disclosure relates to compositions and methods for vaccinating susceptible individuals against infection with P. vivax by administering particulate vaccines comprising P. vivax structural components or variants thereof, and optionally an adjuvant.

Plasmodium vivax is a protozoal parasite responsible for malaria disease. P. vivax malaria is a global health problem in many tropical and sub-tropical countries of the world. More than 70% of cases occur in Asia and the Americas. An effective vaccine that provides protection and prevents transmission is considered the most cost-effective tool for malaria control and would greatly facilitate P. vivax elimination.

The best targets for malaria vaccine development are parasite antigens that can induce an effective immune response in natural and experimental infections that are capable of inhibiting host cell invasion and parasite development. Promising candidates are parasite proteins that play an important role in targeting host cell infection.

The P. vivax circumsporozoite surface protein (CSP), the most abundant molecule on the sporozoite surface, is a leading pre-erythrocytic (PE) vaccine candidate and a prime target in irradiated sporozoite immunity.

CSP is essential for sporozoite traversal of Kupffer cells and entry into the liver parenchyma. Anti-CSP antibodies can prevent sporozoite migration and infection of hepatocytes. These features make CSP a prime target for a PE stage vaccine. Limitations of conventional vaccines such as low immunogenicity, toxicity, instability and the need for multiple doses of conventional vaccines is a challenge for vaccine efficacy. Innovative technologies including nanoparticle-based vaccines show great potential as an alternative to conventional sub-unit vaccines.

The P. vivax CSP is characterized by relatively conserved N-terminal and C-terminal domains which are important for hepatocyte binding and invasion respectively, and an immunodominant polymorphic central repeat region (CRR). Based on the CRR, two major and distinct strains of P. vivax CSP (CSP VK210 and CSP VK247) have been identified in endemic regions. Most of the protection observed with CSP vaccines is associated with the immunodominant CRR.

The P. vivax Belem strain is another variant commonly found in endemic regions.

### SUMMARY

Research into P. vivax vaccine targets have focused mainly on blood stage antigens, with very few studies on pre-erythrocytic (PE) stage antigens.

Sporozoites, the infective stage of the malarial parasite are considered ideal targets for antimalarial strategies and protective immunity. Sporozoites constitute a bottleneck in the complex life cycle of the protozoal parasite. As only a very few sporozoites are injected by an infected mosquito, they tend to have a longer time of exposure to the host immune system than blood-stage antigens, and this stage is clinically silent in a subject.

Liver infection is an obligatory step in malarial transmission. Once injected into the skin, sporozoites actively migrate in the dermis, traverse the capillary epithelium into the bloodstream through the liver sinusoids into the parenchyma where they invade host hepatocytes, proliferate, and develop into exoerythrocytic forms (EEFs) inside a parasitophorous vacuole. Thus, current PE vaccines are aimed at targeting the sporozoites and the EEFs, thereby preventing progression of the parasite to the blood stage.

Studies have demonstrated that subunit vaccines based on sporozoite surface antigens and attenuated whole sporozoites can induce protection in both animal models and humans. Orthologues of sporozoite antigens from other Plasmodium spp are also present in P. vivax and have been shown to play critical roles during hepatocyte infection.

The P. vivax circumsporozoite surface protein (CSP), the dominant molecule on the sporozoite surface, is a leading PE vaccine candidate and a prime target in irradiated sporozoite immunity. CSP plays multiple essential functions throughout pre-erythrocytic stage development including motility, cell traversal, and liver stage development.

Recent studies have demonstrated that CSP-based vaccines can elicit significant protection after sporozoite challenge and attenuate liver-stage (LS) development. Anti-CSP antibodies can prevent sporozoite migration and infection of hepatocytes. CSP forms the basis of the Plasmodium falciparum malaria vaccines currently authorized for use in children in endemic regions. However, very limited progress has been achieved towards a P. vivax CSP based vaccine.

With conventional vaccines, concerns about low immunogenicity, safety, instability, and the need for multiple doses is a challenge. Thus, innovative vaccine formulations and technologies are necessary to combat these diseases.

Recently, nanoparticle-based vaccines (NPVs) have shown great potential for such vaccine formulations. Advances in particle engineering have given the ability to customize composition, particle size, surface characteristics, shape and biodegradability, making it possible to express adjuvants and antigens on the same particle. This offers nanoparticles great potential as a suitable platform for co-delivery of antigens and adjuvants to the immune system.

The relatively small size of NPVs and the fact that antigens can be displayed in a repetitive, ordered array on their surface that mimics the surface of pathogens, facilitates an increased innate immune activation, improved trafficking directly to draining lymph nodes, enhanced antigen uptake by antigen presenting cells, stronger affinity for B-cell receptors due to cross linking of B-cell receptors, and enhanced T-cell help in driving B-cell activation.

Antigens can be incorporated into NPVs by conjugation or encapsulation, which enhances not only antigen stability and immunogenicity, but also targeted delivery and sustainable antigen release to enhance immune exposure.

As P. vivax CSP antigen elicits protective immunity against pre-erythrocytic parasites if delivered with a suitable vaccine platform, nanoparticles described herein are a suitable platform for PE stage vaccine delivery.

In one aspect, disclosed herein are nanoparticles as a platform for co-delivery of recombinant P. vivax CSP antigens and adjuvants directly, to target lymphoid tissues and immune cells for enhanced immune stimulation and maximum protective efficacy.

In one aspect, disclosed herein is a recombinant CSP nanoparticle vaccine, comprised of the P. vivax CSP full length (CSP_{FL}) and its associated subdomains; the CSP N-terminal (CSP_{NT}), CSP C-terminal (CSP_{CT}), CSP N- and C-terminal (CSP_{NT+CT}) for induction of protective antibodies against sporozoite invasion of hepatocytes.

Another aspect provides a pharmaceutical composition comprising a PLGA based nanoparticle and a pharmaceutically acceptable carrier.

Another aspect provides a PLGA based nanoparticle or a pharmaceutical composition for use in treatment or prevention of malaria disease.

Another aspect provides a method for inducing an immune response against P. vivax in a subject, said method comprising administering to the subject an immunologically effective amount of the PLGA based nanoparticle or a pharmaceutical composition as described herein.

Another aspect provides a method of treating or preventing malarial disease in a subject in need of such treatment or prevention, the method including administering a therapeutically or prophylactically effective amount of CSP to the subject.

Another aspect provides a method for immunizing a subject susceptible to a malarial disease, the method including administering a recombinant CSP nanoparticle vaccine provided herein including embodiments thereof to the subject, under conditions such that antibodies directed to the P. vivax circumsporozoite surface protein or a fragment thereof are produced.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIGS. 1A-1B show a schematic of full length CSP-P01 and its subdomains. FIG. 1A shows P. vivax full length CSP gene structure (CSP_{FL}) with the N-terminal, central repeat and C-terminal regions, signal peptide (SP), region 1 (RI), Region III (RIII), the TSR domain and GPI anchor. The arrow represents regions of the gene expressed as a recombinant protein. FIG. 1B shows peptide sequences of central repeat regions and random peptide-1 used for immunizations.

FIG. 2 shows Coomassie stained SDS-PAGE gels of purified recombinant P. vivax CSP full length subdomains. Recombinant proteins are expressed in E. coli (DE3) Star™ and purified by affinity chromatography.

FIG. 3 shows optimized nanoparticle vaccine (NPV) production workflow and quality control.

FIGS. 4A-B show antigen-NP conjugation methods. Size controlled nanoparticles were created by flash nanoprecipitation (FNP) as biodegradable poly(lactic-co-glycolic acid) (PLGA) particles of <100-nm. Multiple copies of antigen are conjugated on the NP surface. These conjugation methods leave key epitopes unperturbed. FIG. 4A shows recombinant CSP modified with Biotin-PEG-SH via an N-terminal AviTag™ on the protein to yield terminal cysteines for direct conjugation to the Mal-NP. FIG. 4B shows how peptides are conjugated to NP via a C-terminal cysteine (SH) group.

FIGS. 5A-E show immunogenicity of CSP-subdomain nanoparticle vaccine antigens. NPVs were tested in duplicate by end point titer ELISA for reactivity with the homologous recombinant antigens. Each curve represents individual mouse serum and error bars represent standard deviation. FIG. 5A shows immune sera from BALB/c mice immunized with recombinant CSP_{FL}. FIG. 5B shows immune sera from BALB/c mice immunized with recombinant CSP_{NT}.

FIG. 5C shows immune sera from BALB/c mice immunized with recombinant CSP_{CT}. FIG. 5D shows immune sera from BALB/c mice immunized with recombinant and CSP_{NT+CT}. FIG. 5E shows immune sera tested at 1:10,000 dilution. Each point represents individual mouse serum with median reactivity and interquartile range.

FIGS. 6A-B show immunogenicity of CSP-subdomain nanoparticle vaccine antigens. FIG. 6A shows Immune sera from BALB/c mice immunized with recombinant CSP_{FL}-P01, CSP_{NT} and CSP_{CT} and CSP_{NT+CT} were tested by end point titer ELISA for reactivity with rCSP_{FL} (recombinant CSP_{FL}). Each curve represents individual mouse serum and error bars represents standard deviation. FIG. 6B shows the immune sera tested at 1:10,000 dilution. Each point represents individual mouse serum with median reactivity and interquartile range.

FIGS. 7A-D show immunogenicity of CSP-NPVs. Each mouse serum was tested by end point dilution starting at 1:500 dilution. FIG. 7A shows the reactivity of immune sera raised in mice against full length recombinant CSP-P01 VK210 NPV with homologous CSP_{FL}-P01 VK210.

FIG. 7B shows the reactivity of immune sera raised in mice against full length recombinant CSP-P01 VK210 NPV with heterologous CSP_{FL}-Belem. FIG. 7C shows the reactivity of immune sera raised in mice against full length recombinant CSP-P01 VK210 NPV with heterologous CSP_{FL}-P01 VK247. FIG. 7D shows the reactivity of each immune sera compared at 1:10,000 dilution.

FIGS. 8A-B show the reactivity of anti-CSP subdomain immune sera with native sporozoite antigen. FIG. 8A shows an immunofluorescence assay (IFA) with cryopreserved P. vivax sporozoites. FIG. 8B shows an immunoblot analysis showing reactivity of anti-CSP subdomain immune sera with sporozoite extracts from cryopreserved P. vivax CSP-VK210 sporozoites (PvCSP Spz), transgenic P. berghei-PvCSP-P01 VK210 sporozoites (Pb-PvCSP Spz) and rPvCSP-P01 VK210 (rPvCSP). Anti-PvCSP-VK210 specific monoclonal antibody (mAb 2F2) served as positive control.

FIGS. 9A-B show the reactivity of CSP NPV antibodies with native sporozoite protein. FIG. 9A shows IFA where antibodies recognize native CSP on the surface of transgenic P. berghei sporozoites expressing P. vivax CSP P01 (Pb-PvCSP-P01). FIG. 9B shows an immunoblot analysis of the antibodies' reactivity with native CSP in crude sporozoite extract of P. vivax sporozoites (VK210 repeats), Pb-PvCSP-P01 VK210 sporozoites, wildtype P. berghei sporozoites and rPvCSP P01 VK210.

FIGS. 10A-F show the generation and characterization of Pb-PvCSP P01 transgenic parasite line. FIG. 10A shows a schematic representation of the generation of Pb-PvCSP replacement line (line Pb05cl1). Pb-Pvcsp P01 replacement construct (SK052) was used to replace the positive negative hdhfr::yfcu selectable marker (SM) cassette in the PbΔcsp-GIMO line, resulting in the generation of Pb-PvCSP replacement line (line Pb05cl1) after negative selection with 5-Fluorocytosine (5-FC). The linearised Pvcsp replacement construct integrates by double cross-over homologous recombination using Pbcsp homology regions resulting in the introduction of the Pvcsp open reading frame (orf) at Pbcsp locus. Black arrows indicate the location and primer number used for diagnostic PCR. FIG. 10B shows genotyping of Pb-PvCSP replacement line by diagnostic PCR analysis. Diagnostic PCR analysis confirms the correct integration of the Pvcsp orf in the Pbesp locus. Correct integration is shown by the presence of Pvcsp orf, correct integration of the targeting construct into Pbcsp locus at both the 5′ and 3′ regions (5′int and 3′int) and the absence of the hdhfr::yfcu SM (see FIG. 10A for primer numbers and locations). Primer sequences and expected PCR product sizes are shown in Table 1. FIG. 10C shows a dot plot with the number of oocysts from individual Anopheles stephensi mosquitoes (median+/−95% confidence interval) infected with Plasmodium berghei wild type (PbWT) or Pb-PvCSP replacement line (n=23). No significant (ns) difference was observed between the oocyst numbers of PbWT and Pb-PvCSP replacement line (Mann-Whitney test, p=0.6672). FIG. 10D shows the average number of salivary gland sporozoites produced in mosquito (each dot represents an experiment with 80-100 mosquitoes) infected with PbWT and Pb-PvCSP replacement line. Bar represents mean and standard deviation. (Mann-Whitney test, **p=0.0063). FIG. 10E shows sporozoite infectivity of Pb-PvCSP transgenic line in Swiss mice. The dot plot shows the parasite liver loads in mice infected with PbWT or Pb-PvCSP P01 sporozoites expressed as relative luminescence units (RLU). Significance of RLU values (Mann-Whitney test): not significant (ns). FIG. 10F shows a Kaplan-Meier curve of the prepatent period of mice infected with 1×10⁴ sporozoites of either PbWT or Pb-PvCSP P01 parasite lines. Significance values of day to patency [Log-Rank (Mantel-Cox) test]: not significant (ns).

FIG. 11 shows the functional activity of anti-CSP-P01 NPV immune sera. Sera from mice (n=10) immunized with CSP_{FL}, CSP_{NT} and CSP_{CT} and CSP_{NT+CT} NPVs were evaluated for inhibition of transgenic Pb-PvCSP-P01 sporozoites infectivity of hepatocytes in vitro by ILSDA. Each data point on the graph represents individual mouse serum tested at 1:100 dilution and error bars represent means and standard deviations.

FIGS. 12A-C show the controlled orientation conjugation of peptide antigens using N-terminal cysteines. FIG. 12A shows peptides with a C-terminal cysteine dialyzed into 1×PBS supplemented with conjugation buffer (2 mM EDTA, pH 6.3-6.5). The peptide-SH was then incubated with Mal-NP at a 1.25:1 Mal:SH ratio overnight at 4° C. to yield the peptide-NP nanoparticle vaccine product. This was dialyzed against DDI water prior to addition of 10-20 w/v % sucrose and lyophilization. The peptides met key quality control (QC) parameters of being <100 nm, having a negative zeta potential, high conjugation efficiency (>85%) and successful reconstitution post lyophilization. FIG. 12B shows peptide sequences, with the terminal cysteine residue in bold. FIG. 12C shows key QC parameters.

FIGS. 13A-C show the immunogenicity of CSP monoclonal antibodies (mAbs) with CSP central repeat peptides. ELISA plates coated with polypeptides conjugated to BSA and probed with different concentrations of mAbs specific for either CSP-VK210 or CSP-VK247. FIG. 13A shows reactivity of anti-pvCSP-VK210 antibodies. FIG. 13B shows reactivity of anti-PvCSP VK247 antibodies. FIG. 13C shows peptide sequences used in generating the monoclonal antibodies.

FIG. 14 shows the immunogenicity of NP-CpG conjugated CSP peptides in mice. Immune sera from immunized mice tested against homologous peptide VK210, VK247 and random peptide-2. Each data point represents total IgG antibody level of individual mouse 14 days post third immunization (Day 56). (VK210, n=10; VK247, n=10; and random peptide-2 n=5). Mice immunized with NP-CpG alone serve as controls. Error bars indicate SD. Serum tested at 1:50 dilution.

FIG. 15 shows a sequence alignment of PvCSP-P01 VK210 and VK247. Sequences aligned using PvCSP-P01 VK210 strain as reference. Dot (.) indicates residue is the same as in reference sequence. PvCSP-Belem has VK210 repeats like the P01 strain.

FIG. 16 shows a sequence alignment of PvCSP strains. Sequences were aligned using PvCSP-PV01 VK210 strain as reference. Dot (.) indicates residue is the same as in reference sequence. PvCSP-Belem has VK210 repeats like the PV01 strain.

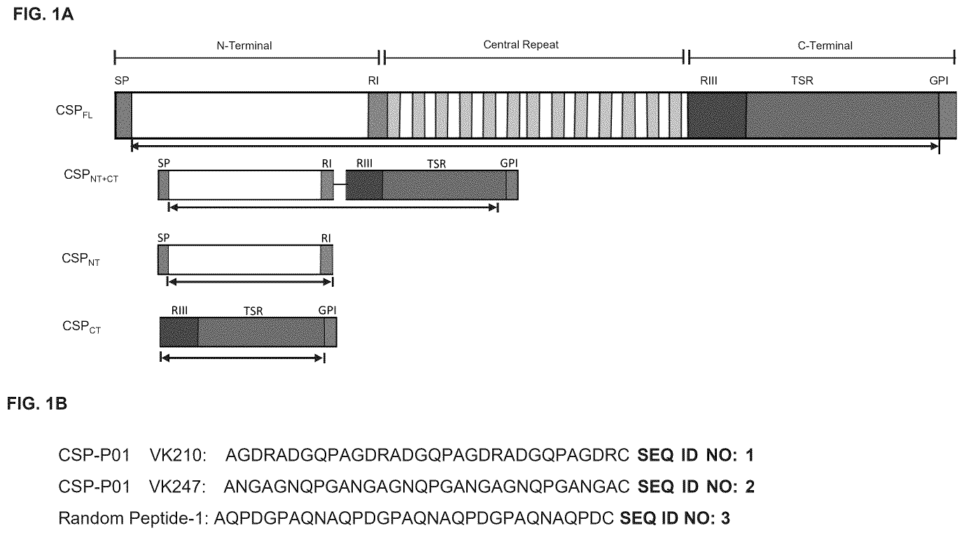

### DEFINITIONS

As used herein, the term “pre-erythrocytic” or abbreviation PE refers to the phase of malaria that occurs in the liver before the parasite infects red blood cells.

As used herein, the phrase “irradiated sporozoite immunity” refers to the immune response developed by a subject when vaccinated with sporozoites that have been exposed to radiation, rendering them unable to fully develop into a mature infection and inducing immunity against malaria disease.

As used herein, the term “ELISA” refers to enzyme-linked immunosorbent assay (ELISA) that uses two antibodies to detect the presence of an antigen.

As used herein, the term “ILSDA” refers to inhibition of liver stage development assay used to measure how well antibodies prevent the development of malaria parasites in the liver of a subject.

As used herein, the term “prepatent period” refers to the time between infection and when a parasite is detectable in the body.

As used herein, the term “malaria disease” refers to the disease caused by a protozoa that is transmitted to a subject by a mosquito.

### DETAILED DESCRIPTION

In a first aspect, provided herein is a synthetic peptide corresponding to P. vivax circumsporozoite peptide-antigen CSP-P01 VK210 comprising SEQ ID NO: 1 or a sequence having at least 90% identity thereto, a synthetic peptide-antigen corresponding to P. vivax circumsporozoite peptide-antigen CSP-P01 VK247 comprising SEQ ID NO: 2 or a sequence having at least 90% identity thereto, and random peptide-1-antigen comprising SEQ ID NO: 3 or a sequence having at least 90% identity thereto, which were used for immunizations.

In another aspect, provided herein is a synthetic peptide-antigen corresponding to P. vivax circumsporozoite protein PvCSP-VK210 comprising SEQ ID NO: 4 or a sequence having at least 90% identity thereto, a synthetic peptide-antigen corresponding to P. vivax circumsporozoite protein PvCSP-VK247 comprising SEQ ID NO: 5 or a sequence having at least 90% identity thereto, and random peptide-2-antigen comprising SEQ ID NO: 6 or a sequence having at least 90% identity thereto, which were used for immunizations.

In another aspect, provided herein is a synthetic peptide-antigen corresponding to P. vivax circumsporozoite peptide AviTag™—CSP-VK210 comprising SEQ ID NO: 7 or a sequence having at least 90% identity thereto, a synthetic peptide-antigen corresponding to P. vivax circumsporozoite peptide AviTag™—CSP-VK247 comprising SEQ ID NO: 8 or a sequence having at least 90% identity thereto, a synthetic peptide-antigen corresponding to P. vivax circumsporozoite peptide AviTag™—CSP-Belem comprising SEQ ID NO: 9 or a sequence having at least 90% identity thereto, a synthetic peptide-antigen corresponding to P. vivax circumsporozoite N-terminal peptide AviTag™—PvCSP-N term comprising SEQ ID NO: 10 or a sequence having at least 90% identity thereto, a synthetic peptide-antigen corresponding to P. vivax circumsporozoite C-terminal peptide AviTag™—PvCSP-C term comprising SEQ ID NO: 11 or a sequence having at least 90% identity thereto, and a synthetic peptide-antigen corresponding to P. vivax circumsporozoite N-plus C-terminal peptide AviTag™—PvCSP-_{N+C} term comprising SEQ ID NO: 12 or a sequence having at least 90% identity thereto, which were used for production of a protein-nanoparticle vaccine. Each peptide contains the AviTag™ sequence as well as a linker sequence.

In another aspect, provided herein is a synthetic peptide corresponding to P. vivax circumsporozoite peptide-antigen CSP-P01 VK210 comprising SEQ ID NO: 1 or a sequence having at least 95% identity thereto, a synthetic peptide-antigen corresponding to P. vivax circumsporozoite peptide-antigen CSP-P01 VK247 comprising SEQ ID NO: 2 or a sequence having at least 95% identity thereto, and random peptide-1-antigen comprising SEQ ID NO: 3 or a sequence having at least 95% identity thereto, which were used for immunizations.

In another aspect, provided herein is a synthetic peptide-antigen corresponding to P. vivax circumsporozoite protein PvCSP-VK210 comprising SEQ ID NO: 4 or a sequence having at least 95% identity thereto, a synthetic peptide-antigen corresponding to P. vivax circumsporozoite protein PvCSP-VK247 comprising SEQ ID NO: 5 or a sequence having at least 95% identity thereto, and random peptide-2-antigen comprising SEQ ID NO: 6 or a sequence having at least 95% identity thereto, which were used for immunizations.

In another aspect, provided herein is a synthetic peptide-antigen corresponding to P. vivax circumsporozoite peptide AviTag™—CSP-VK210 comprising SEQ ID NO: 7 or a sequence having at least 95% identity thereto, a synthetic peptide-antigen corresponding to P. vivax circumsporozoite peptide AviTag™—CSP-VK247 comprising SEQ ID NO: 8 or a sequence having at least 95% identity thereto, a synthetic peptide-antigen corresponding to P. vivax circumsporozoite peptide AviTag™—CSP-Belem comprising SEQ ID NO: 9 or a sequence having at least 95% identity thereto, a synthetic peptide-antigen corresponding to P. vivax circumsporozoite N-terminal peptide AviTag™—PvCSP-N term comprising SEQ ID NO: 10 or a sequence having at least 95% identity thereto, a synthetic peptide-antigen corresponding to P. vivax circumsporozoite C-terminal peptide AviTag™—PvCSP-C term comprising SEQ ID NO: 11 or a sequence having at least 95% identity thereto, and a synthetic peptide-antigen corresponding to P. vivax circumsporozoite N-plus C-terminal peptide AviTag™—PvCSP-_{N+C} term comprising SEQ ID NO: 12 or a sequence having at least 95% identity thereto, which were used for production of a protein-nanoparticle vaccine. Each peptide contains the AviTag™ sequence as well as a linker sequence.

In another aspect, provided herein is a pharmaceutical composition comprising a therapeutically effective amount of at least one of the protein-nanoparticle vaccines disclosed herein, and a pharmaceutically acceptable carrier.

In another aspect, recombinant CSP is formulated as adjuvant and surface conjugated onto PLGA-NPs by controlled orientation conjugation method leveraging the N-terminal AviTag™ on the protein.

In another aspect, provided herein is a method for treating a malaria disease in a subject in need thereof, the method comprising administering to the subject the pharmaceutical composition comprising at least one of the protein-nanoparticle vaccines described herein. In exemplary embodiments, the malaria disease is caused by Plasmodium vivax.

One general aspect includes a nanoparticle vaccine that may include the engineered CSP region or a fragment thereof and a pharmaceutically acceptable carrier.

In another aspect, provided herein are nucleic acids encoding the engineered CSP or a fragment thereof.

In another aspect, provided herein is a method of treating or preventing malaria disease in a subject in need thereof, the method may include administering a therapeutically or prophylactically effective amount of the engineered CSP to the subject.

In another aspect, provided herein is a method for immunizing a subject susceptible to malaria disease, that may include administering the engineered CSP to a subject under conditions such that antibodies are directed to the CSP or a fragment thereof are produced.

In another aspect, provided herein is a method of detecting P. vivax infection in a subject, the method may include: (a) contacting a biological sample obtained from the subject with the engineered CSP or a fragment thereof and (b) determining binding of one or more antibodies to the engineered CSP or a fragment thereof, thereby detecting P. vivax.

The term “identity”, as recognized by those skilled in the art, represents a comparison between two or more amino acid sequences performed using published methods and software known in the art. For example, the compared amino acid sequences are optimally aligned, and the number of amino acid differences are counted and converted to a percentage. For example, if a first amino acid sequence of 50 amino acids is optimally aligned with a second amino acid sequence of 50 amino acids, and 5 out of 50 amino acids differ from the second amino acid sequence, then the first amino acid sequence is said to have 10% identity with the second amino acid sequence. All sequences provided herein may have at least 90%, at least 91%, at least 92%, at least 93%, at least 94%, at least 95%, at least 96%, at least 97%, at least 98%, at least 99%, or 100% identity to the reference sequence.

Inducing a protective response may include inducing immunity against the pathogen, and in some embodiments, inducing protective immunity and/or sterilizing immunity against the pathogen. Inducing a therapeutic response may include reducing the pathogenic load of a subject, for example, as determined by measuring the amount of circulating pathogen before and after administering the composition. Inducing a therapeutic response may include reducing the degree or severity of at least one symptom of infection by the pathogen.

The presently disclosed methods may be utilized for inducing a protective or therapeutic immune response against disease by administering the pharmaceutical compositions disclosed herein (e.g., as immunogenic compositions or vaccines) to a subject in need thereof, which may include a human or non-human having or at risk for acquiring the disease. The methods may include administering a first pharmaceutical composition and optionally may include administering a second pharmaceutical composition to augment or boost an immunogenic response induced by the first pharmaceutical composition. The first and second pharmaceutical compositions may be the same or different. The optionally administered second pharmaceutical composition may be administered prior to, concurrently with, or after administering the first pharmaceutical composition. In some embodiments, the first composition is administered and then the second composition is administered after waiting at least about 1, 2, 3, 4, 5, or 6 weeks. The first composition (and the second composition) may be administered one or more times.

The presently disclosed compositions, kits, and methods may be utilized to protect against or treat infection by a pathogen. As used herein, a “pathogen” includes, but is not limited to a living microorganism such as protozoa, bacteria, virus, and fungi that cause disease in a host. As used herein, a “pathogen” includes Plasmodium vivax.

As used herein, the terms “protein” or “polypeptide” or “peptide” may be used interchangeably to refer to a polymer of amino acids. Typically, a “polypeptide” or “protein” is defined as a longer polymer of amino acids, of a length typically of greater than 50, 60, 70, 80, 90, or 100 amino acids. A “peptide” is defined as a short polymer of amino acids, of a length typically of 50, 40, 30, 20 or less amino acids.

A “protein” as contemplated herein typically comprises a polymer of naturally or non-naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine). The proteins contemplated herein may be further modified in vitro or in vivo to include non-amino acid moieties. These modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine.

The proteins disclosed herein may include “wild type” proteins and variants, mutants, and derivatives thereof. As used herein the term “wild type” is a term of the art understood by skilled persons and means the typical form of an organism, strain, gene or characteristic as it occurs in nature as distinguished from mutant or variant forms. As used herein, a “variant, “mutant,” or “derivative” refers to a protein molecule having an amino acid sequence that differs from a reference protein or polypeptide molecule. A variant or mutant may have one or more insertions, deletions, or substitutions of an amino acid residue relative to a reference molecule. A variant or mutant may include a fragment of a reference molecule. For example, a mutant or variant molecule may one or more insertions, deletions, or substitution of at least one amino acid residue relative to a reference polypeptide.

The term “pharmaceutically acceptable carrier,” as used herein, means a non-toxic, inert solid, semi-solid or liquid filler, diluent, encapsulating material or formulation auxiliary of any type. Suitable pharmaceutically acceptable carriers include, but are not limited to, diluents, preservatives, solubilizers, emulsifiers, liposomes, nanoparticles and adjuvants. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Pharmaceutically acceptable carriers are well known to those skilled in the art and include, but are not limited to, 0.01 to 0.1 M and preferably 0.05M phosphate buffer or 0.9% saline. Additionally, such pharmaceutically acceptable carriers may be aqueous or non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions or suspensions, including saline and buffered media. A tabulation of ingredients listed by the above categories, may be found in the U.S. Pharmacopeia National Formulary, 1857-1859, (1990).

Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator.

Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

The composition may additionally include a biologically acceptable buffer to maintain a pH close to neutral (7.0-7.3). Such buffers preferably used are typically phosphates, carboxylates, and bicarbonates. More preferred buffering agents are sodium phosphate, potassium phosphate, sodium citrate, calcium lactate, sodium succinate, sodium glutamate, sodium bicarbonate, and potassium bicarbonate. The buffer may comprise about 0.0001-5% (w/v) of the vaccine formulation, more preferably about 0.001-1% (w/v). Other excipients, if desired, may be included as part of the final composition. The terms “about” and “approximately” shall generally mean an acceptable degree of error for the quantity measured given the nature or precision of the measurements. Typical, exemplary degrees of error are within 10%, and preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity, such as sodium chloride or dextrose. The pH can be adjusted with acids or bases, such as hydrochloric acid or sodium hydroxide. The preparation can be enclosed in ampoules, disposable syringes or multiple-dose vials made of glass or plastic. For convenience of the patient or treating physician, the dosing formulation can be provided in a kit containing all necessary equipment (e.g., vials of drug, vials of diluent, syringes and needles) for a course of treatment (e.g., 7 days of treatment).

Sterile injectable solutions can be prepared by incorporating the active chemical compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

The preferred route may vary with, for example, the subject's pathological condition or age or the subject's response to therapy or that is appropriate to the circumstances. The formulations can also be administered by two or more routes, where the delivery methods are essentially simultaneous, or they may be essentially sequential with little or no temporal overlap in the times at which the composition is administered to the subject.

Suitable regimes for initial administration and further doses or for sequential administrations also are variable, may include an initial administration followed by subsequent administrations, but nonetheless, may be ascertained by the skilled artisan from this disclosure, the documents cited herein, and the knowledge in the art.

As used herein, the phrase “effective amount” shall mean that dosage that provides the specific immunological response for which a composition comprising that effective amount is administered in a significant number of subjects. For example, an effective amount of an antigen may include that amount which when administered to a vaccinee induces an immune response in the vaccinee, preferably a protective immune response against the pathogen from which the antigen is derived. An effective amount of an antigen that is administered to a particular patient in a particular instance will not always be effective in treating the conditions/diseases described herein, even though such dosage is deemed to be a therapeutically effective amount by those of skill in the art.

The compositions disclosed herein may be formulated as vaccine compositions for administration to a subject in need thereof. Such compositions can be formulated and/or administered in dosages and by techniques well known to those skilled in the medical arts taking into consideration such factors as the age, sex, weight, and condition of the particular patient, and the route of administration.

The compositions may include pharmaceutical solutions comprising carriers, diluents, excipients, and surfactants as known in the art. Further, the compositions may include preservatives. The compositions also may include buffering agents.

The disclosed compositions typically include biodegradable particles. The biodegradable particles typically have an average effective diameter of less than about 100, 90, 75, 50, 25, 10, 1, or 0.5 nm, or the biodegradable particles have an average effective diameter within a range bounded by any of these values (e.g., 0.5-100 nm).

The disclosed biodegradable particles may be prepared by methods known in the art including, but not limited to, spray-drying, precipitation, and grinding. In some embodiments, the biodegradable particles may be formed from a solution or suspension of a biodegradable material optionally in the presence of one or more additional agents such as adjuvants, apoptosis inhibitors, and/or antigens (e.g., by spray-drying the solution or suspension). As such, the biodegradable particles may comprise biodegradable material and optionally may comprise one or more additional agents such as adjuvants, apoptosis inhibitors, and/or antigens.

The disclosed biodegradable particles may be administered by various routes in order to induce a response in a subject. Routes of administration may include, but are not limited to, intranasal, pulmonary, oral, subcutaneous, intramuscular, and intravenous.

In some embodiments, the disclosed methods comprise administering a composition comprising biodegradable particles to induce an immune response in the subject. In other embodiments, the disclosed methods consist of administering a composition consisting of biodegradable particles to induce an immune response in the subject. The induced immune response may include an antibody response, a Th1 cell response and a CD8 CTL response.

The compositions disclosed herein optionally include an adjuvant. The term “adjuvant” refers to a compound or mixture that enhances an immune response. An adjuvant can serve as a tissue depot that slowly releases the antigen and also as a lymphoid system activator that non-specifically enhances the immune response. Examples of adjuvants which may be utilized in the disclosed compositions include but are not limited to, co-polymer adjuvants (e.g., Pluronic L1219 brand poloxamer 401, CRL1005, or a low molecular weight co-polymer adjuvant such as Polygen® adjuvant), poly (I:C), R-848 (a Th1-like adjuvant), resiquimod, imiquimod, PAM3CYS, aluminum phosphates (e.g., AlPO4), loxoribine, potentially useful human adjuvants such as BCG (Bacille Calmette-Guerin) and Corynebacterium parvum, CpG oligodeoxynucleotides (ODN), cholera toxin derived antigens (e.g., CTA1-DD), lipopolysaccharide adjuvants, complete Freund's adjuvant, incomplete Freund's adjuvant, saponin (e.g., Quil-A), mineral gels such as aluminum hydroxide, surface active substances such as lysolecithin, pluronic polyols, polyanions, peptides, oil or hydrocarbon emulsions in water (e.g., MF59 available from Novartis Vaccines or Montanide ISA 720), keyhole limpet hemocyanins, and dinitrophenol.

The compositions disclosed herein may include pharmaceutical compositions that are administered as vaccines. Typically, the pharmaceutical composition comprises an effective amount or concentration of an antigen for inducing a protective or therapeutic immune response against a disease, which may include, but is not limited to infection by a pathogen such as P. vivax. Inducing a protective or therapeutic immune response may include inducing an antibody response, as well as a CD4 and/or CD8 T cell immune response to one or more epitopes of a protein associated with a pathogen (e.g., a protein associated with P. vivax). Inducing a protective or therapeutic immune response may include inducing an antibody response, as well as a Th1 response and/or a CD8 T cell response to one or more epitopes of a protein associated with the pathogen. As utilized herein, a Th1-response may be characterized by cytokine production such as interferons (e.g., IFN-γ), tumor necrosis factor (e.g., TNF), and interleukins (e.g., IL-2). A Th1-response also may be characterized by increased killing efficiency of macrophages with respect to a pathogen and the proliferation of cytotoxic CD8+ cells against the pathogen. A Th1 response also may be characterized by the presence of opsonizing antibodies against the antigen.

In some embodiments, the disclosed particles may be phagocytosed by antigen presenting cells, such as macrophage and dendritic cells, when the disclosed particles are administered as an immunogenic composition or vaccine formulation to a subject in need thereof. Preferably, the disclosed particles have an effective average diameter to permit phagocytosis by antigen presenting cells. Particles larger than about 5 microns are unlikely to be phagocytosed by antigen presenting cells and preferably the particles have an effective average diameter of less than about 4 microns or more preferably the particles have an effective average diameter of less than about 3 microns.

As used herein, the term “administering”, refers to dispensing, delivering, or applying the therapeutic agent, to a subject by any suitable route for delivery of the substance to the desired location in the subject, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

The terms “treating” and “to treat” includes the reducing, repressing, delaying or preventing malaria disease.

As used herein, the term “subject” may be used interchangeably with the term “patient” or “individual” and may include an “animal” and in particular a “mammal.” Mammalian subjects may include humans and non-human animals, such as other primates, domestic animals, farm animals, and companion animals such as dogs, cats, guinea pigs, rabbits, rats, mice, horses, cattle, cows, etc.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter. Embodiments recited as “including,” “comprising,” or “having” certain elements are also contemplated as “consisting essentially of” and “consisting of” those certain elements.

The phrase “such as” should be interpreted as “for example, including.” Moreover the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Methods:

BALB/c mice (6-8 weeks old) were vaccinated subcutaneously with 25 μg antigen/dose at 3 week intervals, with a total of 100 μl/injection at two sites. Test bleeds were done 14 days after each immunization (days 14 and 35). Final bleeds were done 21 days after the second dose. Mice per test group, n=10. Control group, n=5.

### PLGA-b-PEG Nanoparticle Fabrication:

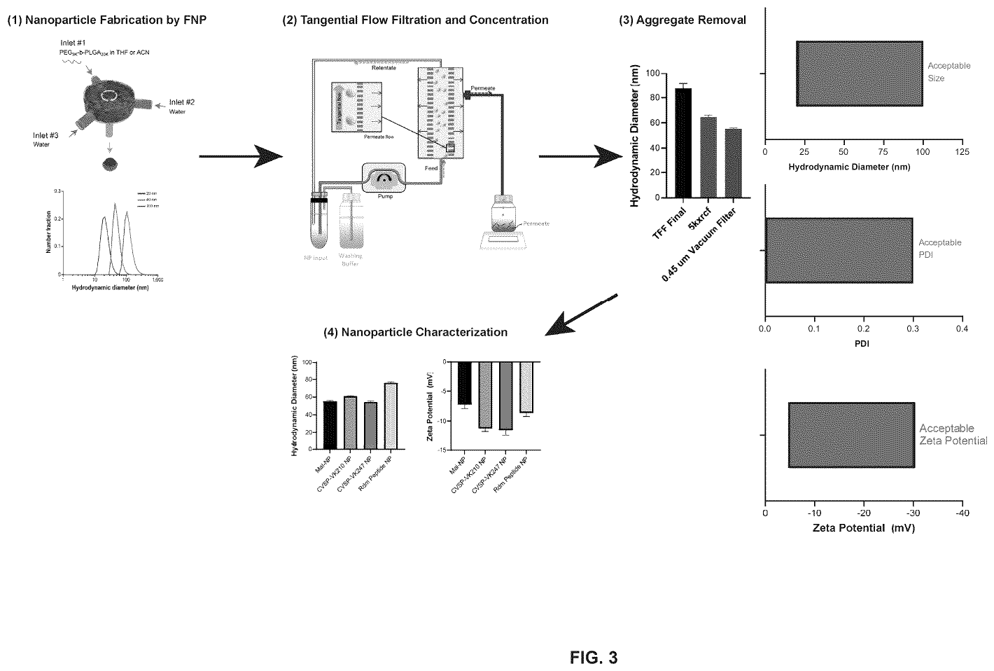

NPs were created by flash nanoprecipitation (FNP) fabricated as biodegradable poly(lactic-co-glycolic acid) (PLGA) particles of uniform small size (<100 nm). Briefly, nanoparticles (NPs) were generated using a three-inlet, confined impinging jet FNP device. Two inlets contained distilled, deionized (DDI) water. The third inlet contained 10 mg/mL PLGA20K-b-mPEG3K with 78.5 mol % PLGA20Kb-mPEG3K and 21.5 mol % PLGA20K-b-PEG3K-Mal dissolved in tetrahydrofuran (THF). NPs of defined size were generated by modulating volumetric flow rates of the three input inlets using a NE-4000 Programmable 2 Channel Syringe Pump (New Era Systems, Inc., Farmingdale, NY, USA). For 30-40 nm NPs, a flow rate of 22 mL/min was used for all three inlets. The NPs generated through the device were collected in a water bath such that the total organic solvent was less than 10% v/v. The collected NPs were dialyzed against 4 L of DDI water in a 3.5-kDa MWCO dialysis membrane at 4° C. with dialysate changes every 6 h for 18-24 h. The final NP was then characterized using Malvern Zetasizer Nano ZS dynamic light scattering (DLS) to measure the NP size and surface charge (zeta potential). The NPs were then lyophilized and stored and stored at −20° C. (FIG. 3).

Nano particles were comprised of biodegradable poly(lactic-co-glycolic acid) (PLGA) nanoparticles, with particle size <100 nm. Nanoparticle vaccine (NPV) was created by flash nanoprecipitation (FNP). Antigen was formulated with CpG-1018 and surface conjugated to PLGA-NPs (PLGA-nanoparticles).

### Recombinant CSP Antigen Production:

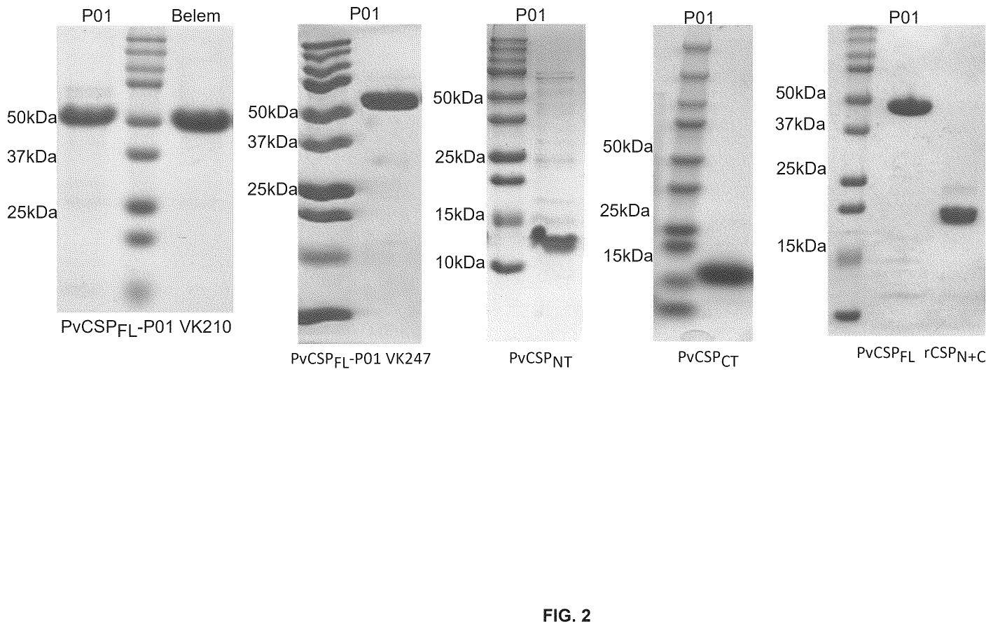

The ORF of the gene sequence coding for full length P. vivax CSP (CSP_{FL}) of the P01 strain (PVP01_0835600.1), and its subdomains (excluding the signal peptide and GPI anchor sequences) were codon optimized for E. coli expression, commercially synthesized, and cloned into an expression vector (pET21a+) with a C-terminal 6× His-tag to facilitate purification by affinity chromatography, and an N-terminal AviTag™ sequence to aid recombinant protein conjugation to nanoparticles (NP). Recombinant proteins were expressed in E. coli BL-21 Star (DE3) chemically competent strain (Invitrogen) and purified on HisTrap HP columns (Cytiva) using the AktaPure system (GE HealthCare). Purified proteins were validated by SDS-PAGE chromatography (FIG. 2). Peptides representing the central repeat regions (CRR) of CSP VK210 and CSP VK247 and a random peptide-1 (control) were obtained through a commercial vendor (ABclonal, USA).

TABLE 1
List of primers
Primers used to generate targeting construct SK052

| Sequence | Primer description |
|---|---|
| SEQ. ID. NO: 16 AAGCTTGGGCCCTCGAGCAAGCATATACATAAAAGGGAATATGG | Pbcsp 5′HR forward |
| SEQ. ID. NO: 17 GTCGACTTTAAATATATGCGTGTATATATAGATTTTGTTTTTTG | Pbcsp 5′HR reverse |
| SEQ. ID. NO: 18 CGTCGACATAAACATTACGCATGATTATAAATATTTATA | Pbcsp 3′HR forward |
| SEQ. ID. NO: 19 GAATTCTCGAGAATTAACCAATGCTGTATACTATTATAATGTC | Pbcsp 3′HR reverse |

Primers used for integration PCRs of Pb-PvCSP P01 (Pb05cl1)

| Sequence | Primer description | Amplicon |
|---|---|---|
| SEQ. ID. NO: 20 CCAACACACTG | Pvcsp orf forward | Pvcsp orf |
| TGGTCACAACG | | |
| SEQ. ID. NO: 21 CAACGTTAAAT | Pvcsp orf reverse | Pvcsp orf |
| ATACCGGCACACTTATCC | | |
| SEQ. ID. NO: 22 CAATTCCTCTT | Pbcsp integration forward | 5′ integ. |
| TCAATCTGTACATATTTATTCTC | | |
| SEQ. ID. NO: 23 TCTACGTTGTG | Pvcsp integration reverse | 5′ integ. |
| ACCACAGTGTGTTG | | |
| SEQ. ID. NO: 24 CATTGAATGAC | Pvcsp integration forward | 3′ integ. |
| CTTGAGACTGATGTTTGTAC | | |
| SEQ. ID. NO: 25 GTATTTTTATT | Pbcsp integration reverse | 3′ integ. |
| GAAAAAGACACAAAATAGCTAGTTTG | | |
| SEQ. ID. NO: 26 GGTATTCCACG | SM forward | Absence of SM |
| CTGTGTTGTCGGTGAG | | |
| SEQ. ID. NO: 27 GTTCATAATTT | SM reverse | Absence of SM |
| TAGCTATTTACATGCATGTGC | | |
| SEQ. ID. NO: 28 ATGAAGAAGTG | Pbcsp orf forward- | Absence of |
| TACCATTTTAGTTGTAGCG | | Pbcsp orf |
| SEQ. ID. NO: 29 GTATCAATATC | Pbcsp orf reverse- | Absence of |
| TTCTAAGGTCAAATCTTCTGC | | Pbcsp orf |

*HR - homologous region
*orf - open reading frame
*SM - selectable marker

### Conjugation of Recombinant CSP to Nanoparticles

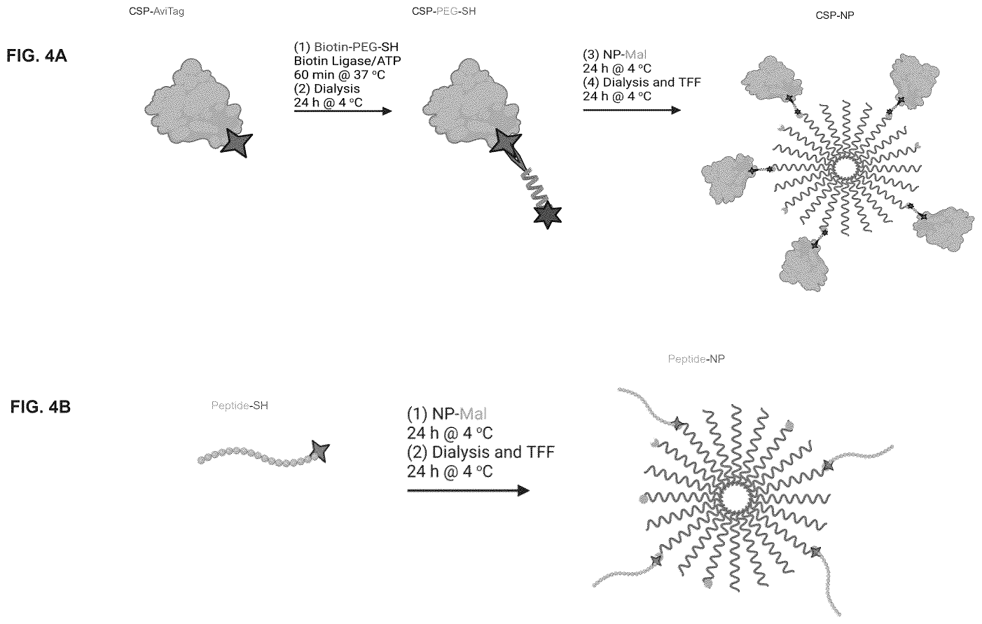

A versatile and efficient production platform for conjugation of various antigen constructs onto PEG-PLGA NPs with precise control over the size and surface presentation of the antigens was used. Recombinant CSP was formulated in CpG-1018 as adjuvant and surface conjugated onto the PLGA-NPs by a controlled orientation conjugation method leveraging the N-terminal AviTag™ on the protein (FIG. 4A). First, the AviTag™ protein is biotinylated with Biotin-PEG-SH to yield an N-terminal cysteine with a thiol group (SH). This was achieved by incubating 50 μM of Biotin-PEG-SH with 10 mM ATP, 10 mM MgCl₂, 6.4 ng/μL biotin ligase, and biotin ligase buffer for every 40 μM of protein substrate at 37° C. for either 3 h or overnight. The degree of Biotin-PEG-SH modification of the AvitTag™ protein was quantified using a HABA assay according to the manufacturer's instructions (Thermo Scientific). The resulting protein-SH product was then dialyzed in PBS supplemented with 2 mM EDTA (pH 6.3-6.5), with three buffer changes every 4 hours in a D-Tube™ Dialyzer Mini MWCO 12-14 kDa dialysis tube (Millipore Sigma). The protein was then conjugated to the NP through the thiol group using thio-maleimide chemistry, by incubating the modified protein with Mal-NP at a 1.25:1 Mal:SH ratio overnight at 4° C. to yield the protein-nanoparticle vaccine (protein-NPV) product. The resulting product was then dialyzed against DDI water with three dialysis buffer changes in a 1-kDa MWCO dialysis tubing (Repligen) every 4 h prior to addition of 10-20 w/v % sucrose and lyophilization.

Peptides were Conjugated to NPs by a Controlled Orientation Conjugation of Leveraging Thiol Groups of C-Terminal Cysteines

Peptides with an N-terminal cysteine were dialyzed into 1×PBS supplemented with 2 mM EDTA (pH 6.3-6.5) conjugation buffer with three dialysis buffer changes every 4 h in a D-Tube™ Dialyzer Mini MWCO 12-14 kDa dialysis tube (Millipore Sigma). The peptide-SH was then incubated with Mal-NP at a 1.25:1 Mal:SH ratio overnight at 4° C. to yield the peptide-NP nano vaccine product (FIG. 4B). This was dialyzed against DDI water with three dialysis buffer changes in a 1-kDa MWCO dialysis tubing (Repligen) every 4 h prior to addition of 10-20 w/v % sucrose and lyophilization.

### Immunogenicity of NPVs in Mice

Immunogenicity of the NPVs were evaluated in 6-8 week old female BALB/c mice. Briefly, groups of mice were immunized twice subcutaneously (sc) at three week intervals with 25 μg of full length recombinant CSP-P01 VK210 (CSP_{FL}) or its related subdomains, N-terminal CSP (CSP_{NT}), C-terminal CSP (CSP_{CT}), combination of CSP N and C terminus without the repeats (CSP_{NT+CT}) NPV or peptide NPV. Control mice received only NP+adjuvant in PBS or random peptide conjugated to NP. Test sera were collected 14 days after each immunization dose and final bleeds for serum collected by cardiac puncture three weeks after the final immunization dose. All sera were stored at −20° C. until needed.

### CSP Specific Antibody Responses

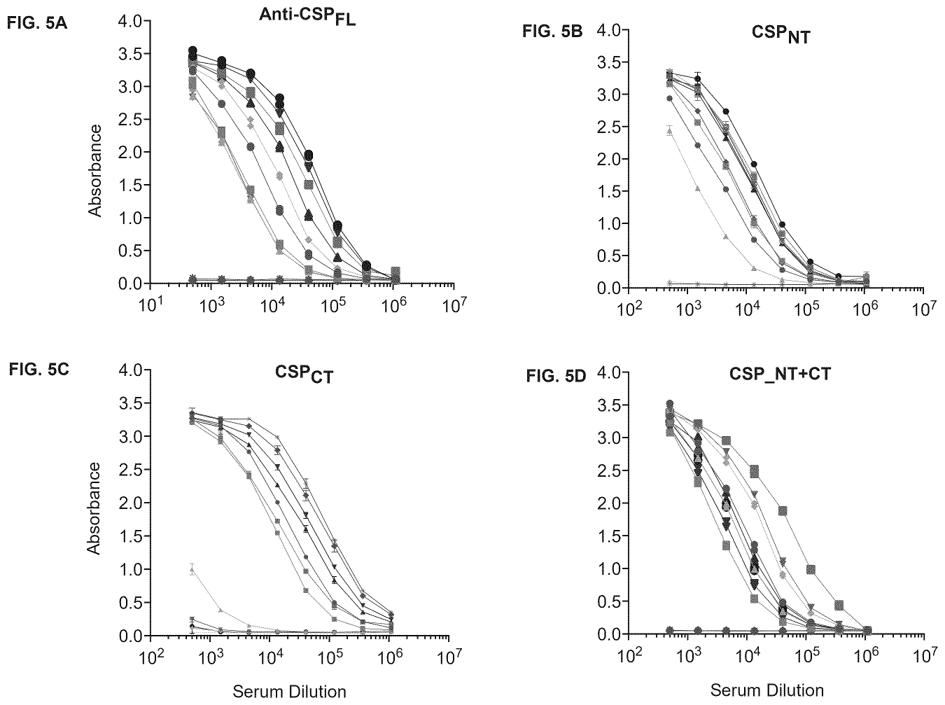

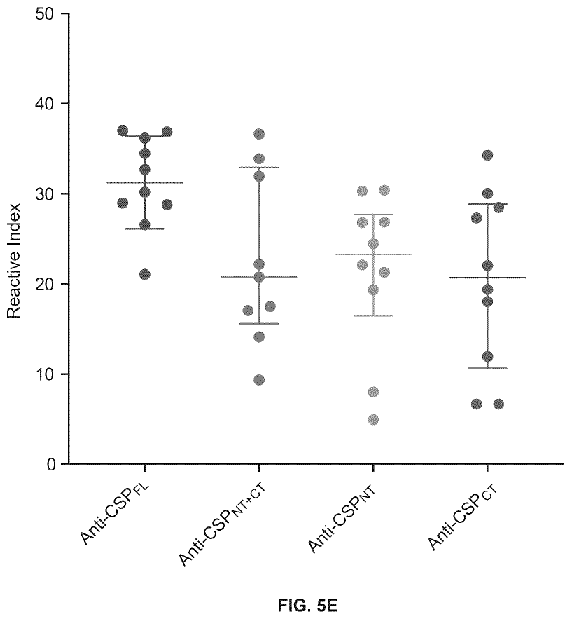

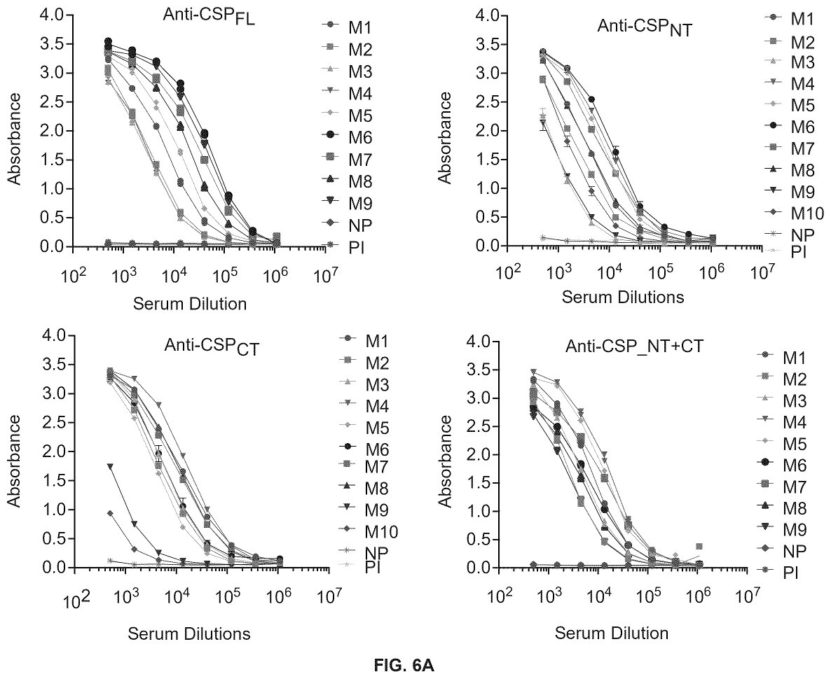

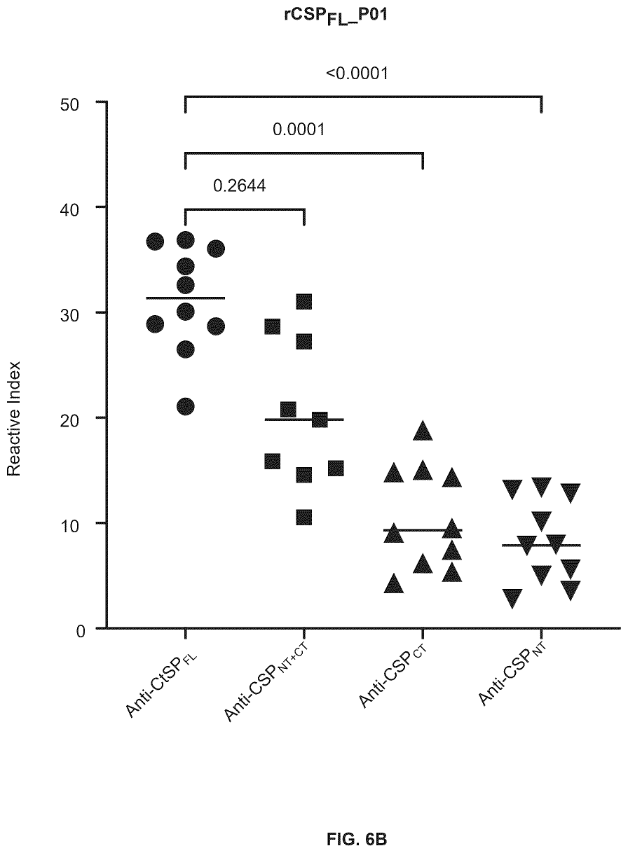

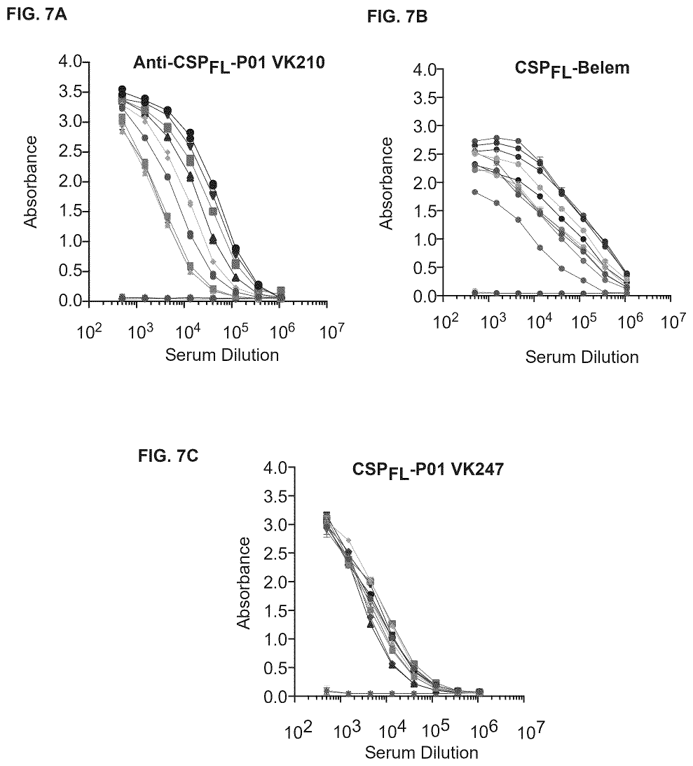

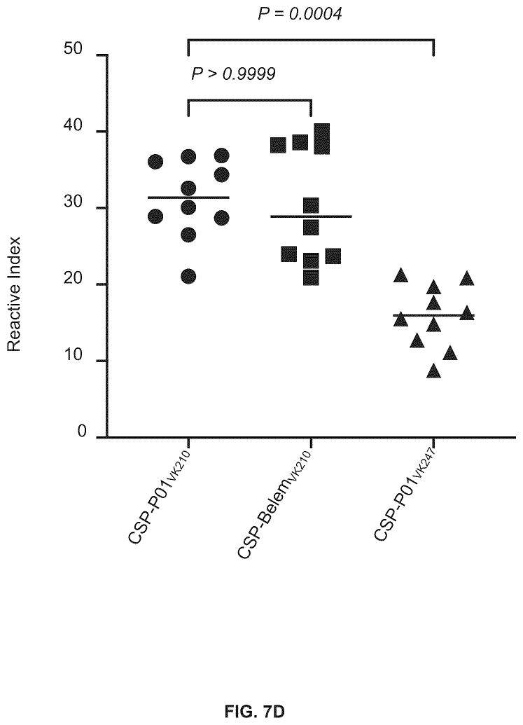

The immunogenicity of the different CSP-NPVs was evaluated in BALB/c mice. Total IgG antibody titers in sera of immunized mice were analyzed by standard ELISA against the homologous rCSP antigens. All the vaccine antigens induced high titer IgG antibodies against the respective recombinant proteins (FIGS. 5A-D), with the CSP_{FL} inducing a relatively higher antibody response compared to the subdomains individually or in combination (FIG. 5E). There was considerable variability in antibody responses induced by the individual mice. The CSP subdomain immune sera were also tested for cross-reactive antibodies against full-length rCSP-P01 VK210 (FIG. 6). The CSP_{FL} VK210 immunized mice induced significantly higher IgG antibody responses against rCSP_{FL} VK210 than the individual CSP_{NT} and CSP_{CT} antigens (p<0.0001) but not against the rCSPNT+CT (p=0.2644) (FIG. 6). Similarly, the CSP_{NT+CT} antigen induced a significantly higher IgG antibody response than the individual all the CSP_{NT} and CSP_{CT} (p<x) (FIG. 6). Anti-CSPFLP01 VK210 immune sera also cross reacted with heterologous rCSP-Belem and rCSP-P01 VK247 (FIG. 7). There was no significant difference in reactivity between rCSP_{FL}-P01 VK210 and rCSPFLBelem (p=0.999) and a significantly lower reactivity with rCSP-P01 VK247 (p=0.004).

### Anti-CSP Antibodies Recognize Native CSP on Sporozoites

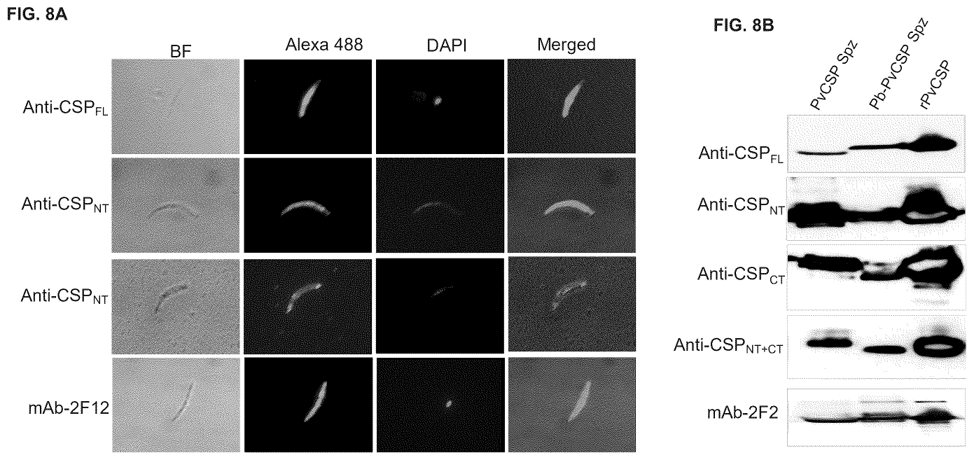

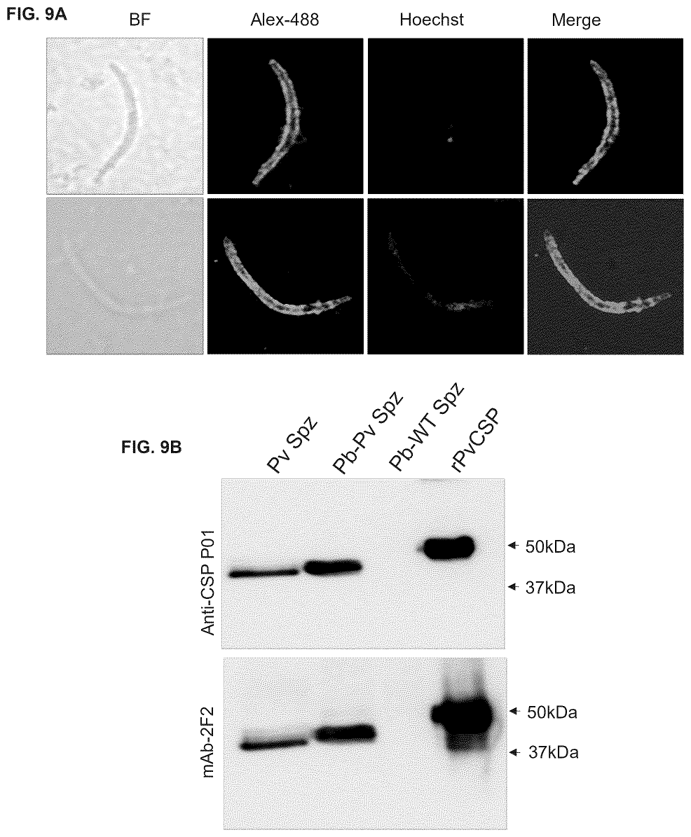

Immune sera were also tested for reactivity with native parasite proteins by IFA and Western blot analysis. NPV immune sera reacted with the native antigen on cryopreserved P. vivax sporozoites and transgenic P. brghei parasites expressing PvCSP-P01 VK210 by IFA and western blots analysis (FIGS. 8A-B and FIGS. 9A-B).

### Results

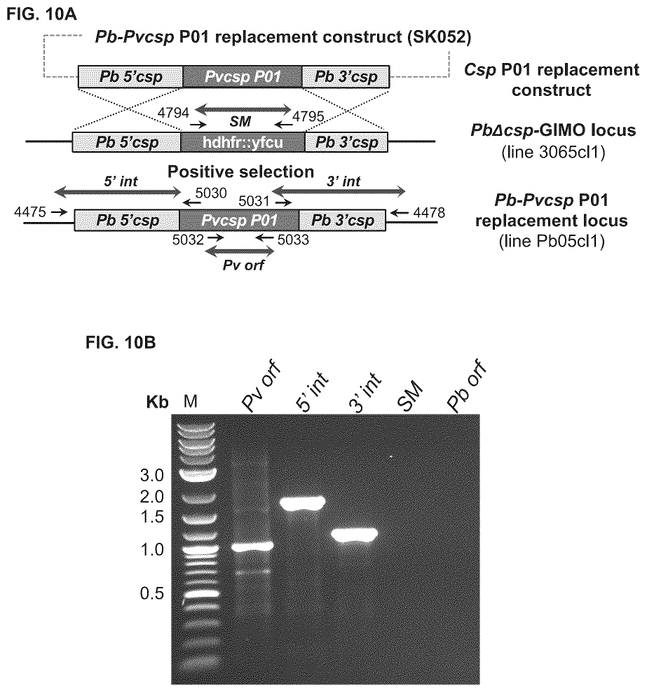

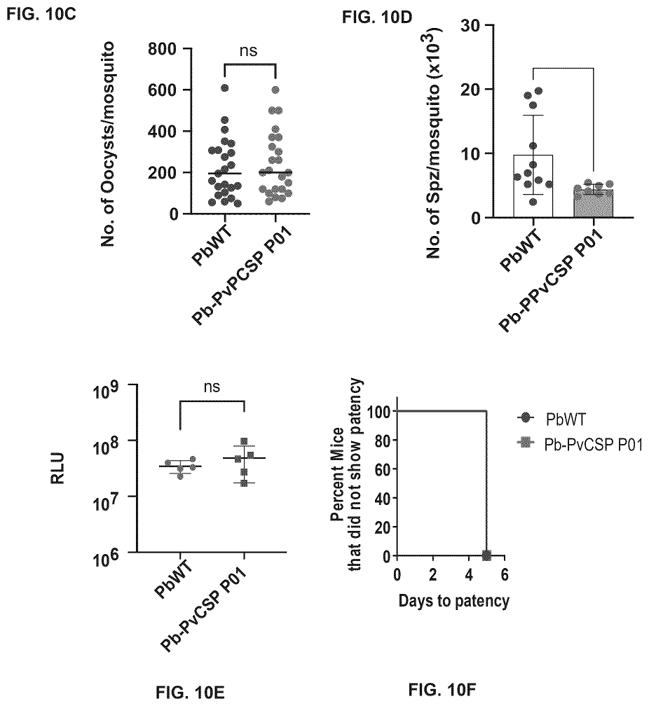

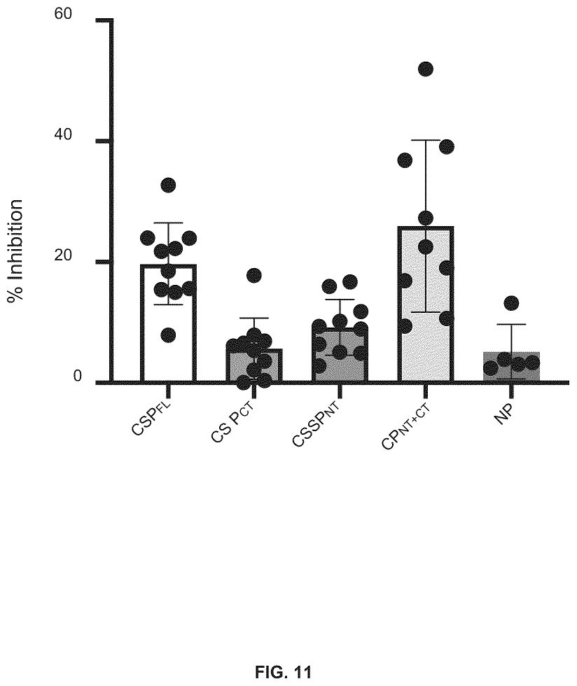

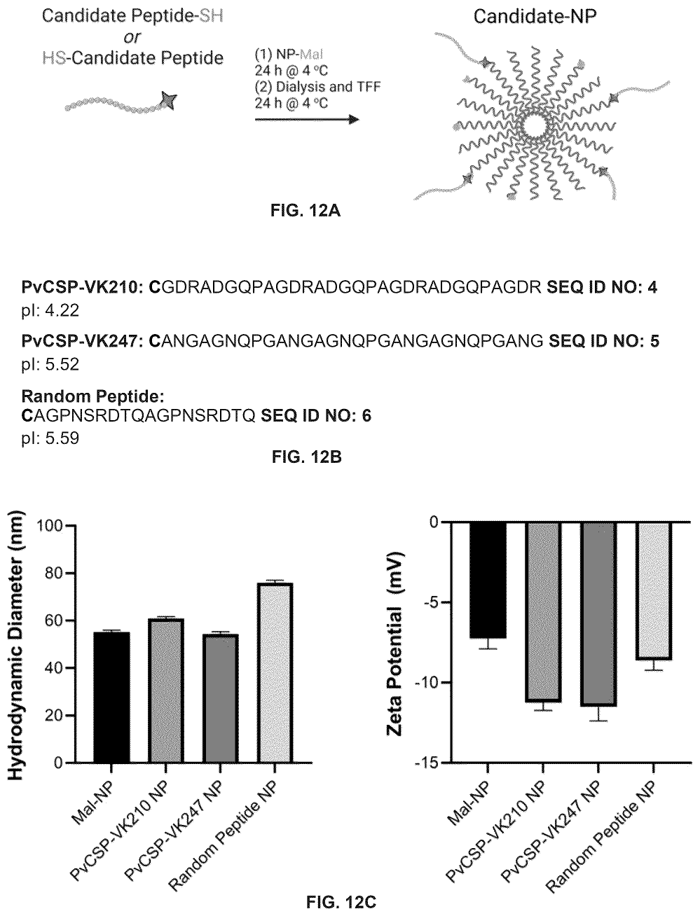

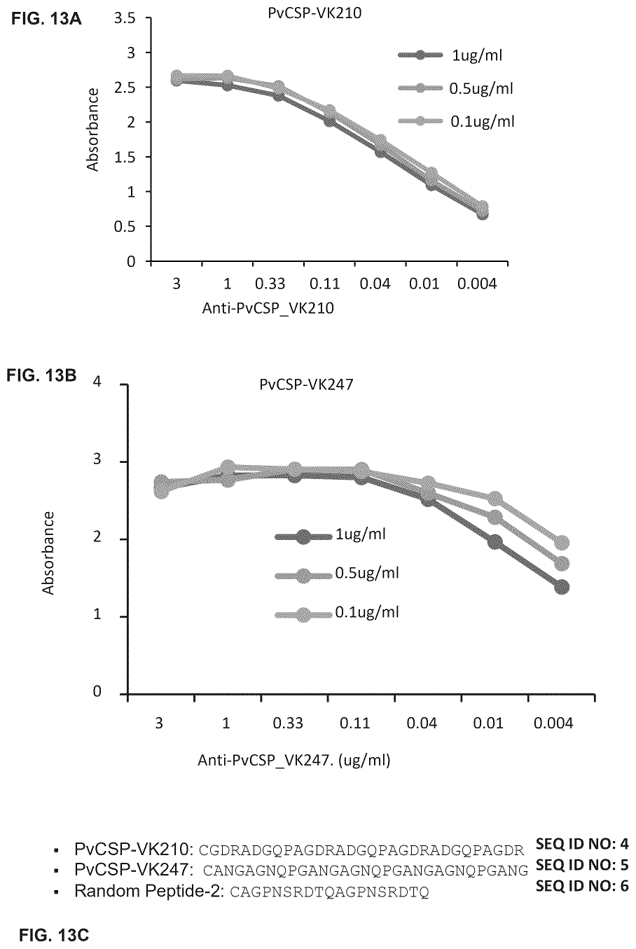

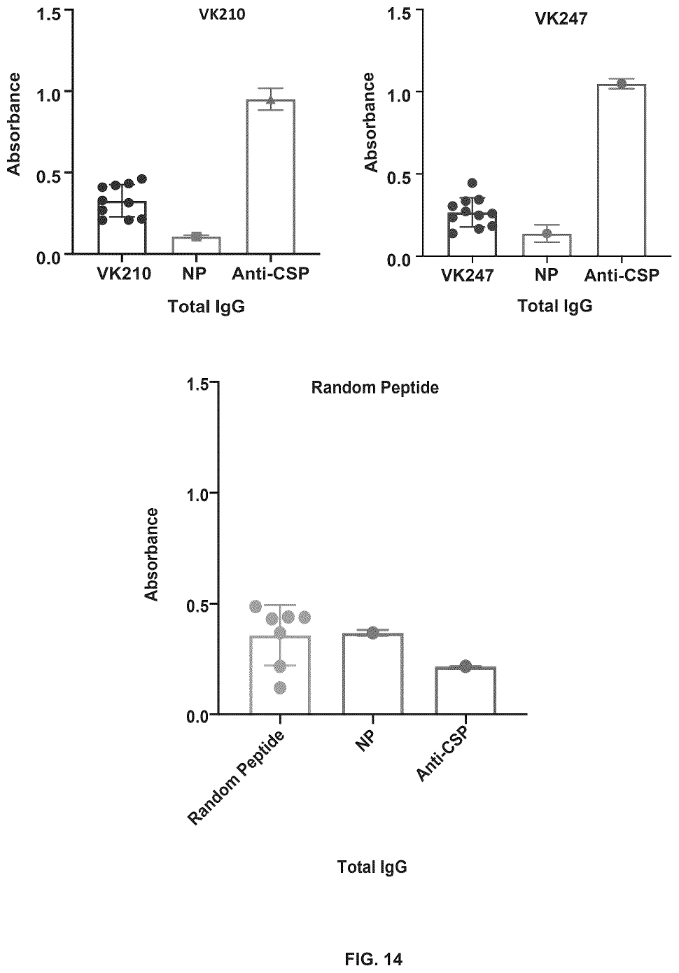

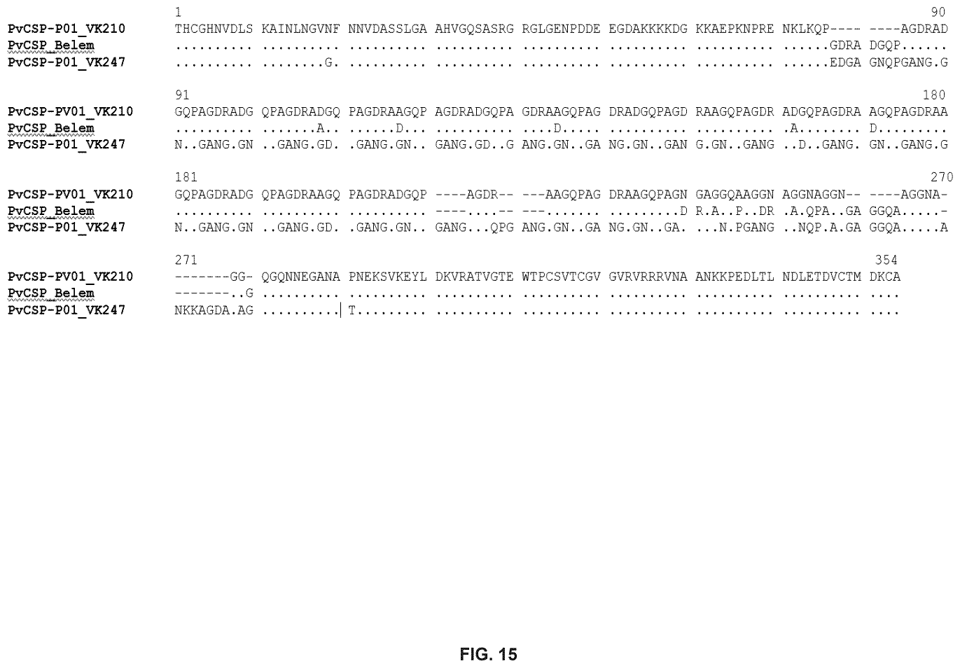

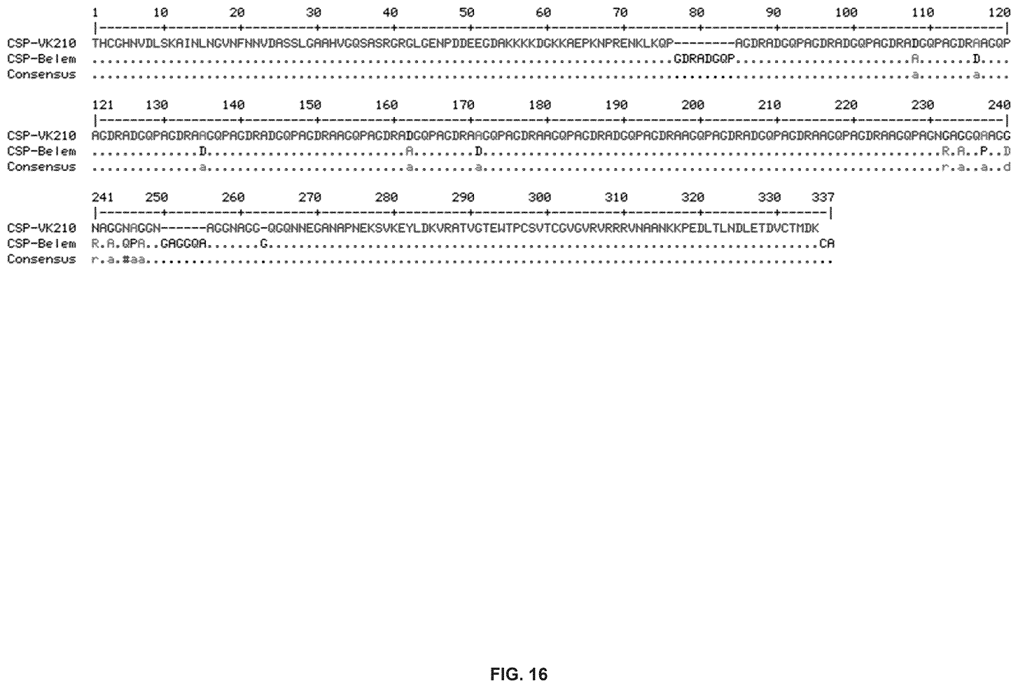

The CSP NPV immune sera was evaluated for inhibition of sporozoite invasion of hepatocytes (HC-04 cells) and liver stage development in an in vitro inhibition of liver stage development assay (ILSDA). In the absence of P. vivax sporozoites, we generated a transgenic P. berghei parasite line, which expresses P. vivax CSP-P01 VK210 in the place of endogenous P. berghei CSP (FIG. 10A). This assay serves as a useful surrogate assay to study P. vivax liver stage infection and development. The data shows that all of the CSP NPV immune sera inhibit sporozoite invasion of hepatocytes with variability similar to the ELISA titers (FIG. 11). The anti-CSP_{FL}-P01 VK210 and anti-CSP_{NT+CT} tend to show a similar inhibitory activity against hepatocyte invasion by the sporozoites.

### Methods and Materials

TABLE 1
Informal Sequence Listing

| SEQ ID NO and | |
|---|---|
| Description | Sequence |
| 1 | AGDRADGQPAGDRADGQPAGDRADGQPAGDRC |
| CSP-P01 | |
| VK210 | |
| 2 | ANGAGNQPGANGAGNQPGANGAGNQPGANGAC |
| CSP-P01 | |
| VK247 | |
| 3 | AQPDGPAQNAQPDGPAQNAQPDGPAQNAQPDC |
| Random | |
| peptide-1 | |
| 4 | CGDRADGQPAGDRADGQPAGDRADGQPAGDR |
| PvCSP- | |
| VK210 | |
| 5 | CANGAGNQPGANGAGNQPGANGAGNQPGANG |
| PvCSP- | |
| VK247 | |
| 6 | CAGPNSRDTQAGPNSRDTQ |
| Random | |
| peptide-2 | |
| 7 | GLNDIFEAQKIEWHEGSGSGTHCGHNVDLSKAINLNGVNFNNVDAS |
| AviTag™_CSP- | SLGAAHVGQSASRGRGLGENPDDEEGDAKKKKDGKKAEPKNPREN |
| P01 VK210 | KLKQPAGDRADGQPAGDRADGQPAGDRADGQPAGDRAAGQPAG |
| Full length CSP | DRADGQPAGDRAAGQPAGDRADGQPAGDRAAGQPAGDRADGQP |
| | AGDRAAGQPAGDRAAGQPAGDRADGQPAGDRAAGQPAGDRADG |
| | QPAGDRAAGQPAGDRAAGQPAGNGAGGQAAGGNAGGNAGGNAG |
| | GNAGGQGQNNEGANAPNEKSVKEYLDKVRATVGTEWTPCSVTCG |
| | VGVRVRRRVNAANKKPEDLTLNDLETDVCTMDKCA |
| 8 | GNDIFEAQKIEWHEGSGSGTHCGHNVDLSKAINLNGVGFNNVDASS |
| AviTag™_ | LGAAHVGQSASRGRGLGENPDDEEGDAKKKKDGKKAEPKNPREN |
| CSP- | KLKQPEDGAGNQPGANGAGNQPGANGAGNQPGANGAGDQPGAN |
| P01VK247 | GAGNQPGANGAGDQPGANGAGNQPGANGAGNQPGANGAGNQPG |
| | ANGADDQPGANGAGNQPGANGAGNQPGANGAGNQPGANGAGDQ |
| | PGANGAGNQPGANGAGDQPGANGAGNQPGANGAGNQPGANGAG |
| | NQPGANGAGNQPGANGAGGQAAGGNAANKKAGDAGAGQGQNN |
| | EGANATNEKSVKEYLDKVRATVGTEWTPCSVTCGVGVRVRRRVN |
| | AANKKPEDLTLNDLETDVCTMDKCA |
| 9 | GLNDIFEAQKIEWHEGSGSGPTHCGHNVDLSKAINLNGVNFNNVDA |
| AviTag™_CSP_ | SSLGAAHVGQSASRGRGLGENPDDEEGDAKKKKDGKKAEPKNPRE |
| Belem | NKLKQPGDRADGQPAGDRADGQPAGDRADGQPAGDRAAGQPAG |
| | DRADGQPAGDRADGQPAGDRADGQPAGDRADGQPAGDRAAGQP |
| | AGDRAAGQPAGDRADGQPAGDRAAGQPAGDRADGQPAGDRAAG |
| | QPAGDRADGQPAGDRAAGQPAGDRAAGQPAGDRAAGQPAGDRA |
| | AGQPAGNGAGGQAAGGNAGGGQGQNNEGANAPNEKSVKEYLDK |
| | VRATVGTEWTPCSVTCGVGVRVRRRVNAANKKPEDLTLNDLETDV |
| | CTMDKCA |
| 10 | GLNDIFEAQKIEWHEGSGSGTHCGHNVDLSKAINLNGVNFNNVDAS |
| AviTag™_PvCSP_ | SLGAAHVGQSASRGRGLGENPDDEEGDAKKKKDGKKAEPKNPREN |
| N Term | KLKQP |
| 11 | GLNDIFEAQKIEWHEGSGSGGGQGQNNEGANAPNEKSVKEYLDKV |
| AviTag™_PvCSP_ | RATVGTEWTPCSVTCGVGVRVRRRVNAANKKPEDLTLNDLETDVC |
| C Term | TMDKCA |
| 12 | GLNDIFEAQKIEWHEGSGSGTHCGHNVDLSKAINLNGVNFNNVDAS |
| AviTag™_PvCSP_ | SLGAAHVGQSASRGRGLGENPDDEEGDAKKKKDGKKAEPKNPREN |
| N + C | KLKQPGSGSGGGQGQNNEGANAPNEKSVKEYLDKVRATVGTEWT |
| | PCSVTCGVGVRVRRRVNAANKKPEDLTLNDLETDVCTMDKCA |
| 13 | THCGHNVDLSKAINLNGVNFNNVDASSLGAAHVGQSASRGRGLGE |
| PvCSP-P01 | NPDDEEGDAKKKKDGKKAEPKNPRENKLKQPAGDRADGQPAGDR |
| VK210 | ADGQPAGDRADGQPAGDRAAGQPAGDRADGQPAGDRAAGQPAG |
| | DRADGQPAGDRAAGQPAGDRADGQPAGDRAAGQPAGDRAAGQP |
| | AGDRADGQPAGDRAAGQPAGDRADGQPAGDRAAGQPAGDRAAG |
| | QPAGNGAGGQAAGGNAGGNAGGNAGGNAGGQGQNNEGANAPNE |
| | KSVKEYLDKVRATVGTEWTPCSVTCGVGVRVRRRVNAANKKPED |
| | LTLNDLETDVCTMDKCA |
| 14 | THCGHNVDLSKAINLNGVNFNNVDASSLGAAHVGQSASRGRGLGE |
| PvCSP Belem | NPDDEEGDAKKKKDGKKAEPKNPRENKLKQPGDRADGQPAGDRA |
| | DGQPAGDRADGQPAGDRAAGQPAGDRADGQPAGDRADGQPAGD |
| | RADGQPAGDRADGQPAGDRAAGQPAGDRAAGQPAGDRADGQPA |
| | GDRAAGQPAGDRADGQPAGDRAAGQPAGDRADGQPAGDRAAGQ |
| | PAGDRAAGQPAGDRAAGQPAGDRAAGQPAGNGAGGQAAGGNAG |
| | GGQGQNNEGANAPNEKSVKEYLDKVRATVGTEWTPCSVTCGVGV |
| | RVRRRVNAANKKPEDLTLNDLETDVCTMDKCA |
| 15 | THCGHNVDLSKAINLNGVGFNNVDASSLGAAHVGQSASRGRGLGE |
| PvCSP-P01 | NPDDEEGDAKKKKDGKKAEPKNPRENKLKQPEDGAGNQPGANGA |
| VK247 | GNQPGANGAGNQPGANGAGDQPGANGAGNQPGANGAGDQPGAN |
| | GAGNQPGANGAGNQPGANGAGNQPGANGAADQPGANGAGNQPG |
| | ANGAGNQPGANGAGNQPGANGAGDQPGANGAGNQPGANGAGDQ |
| | PGANGAGNQPGANGAGNQPGANGAGNQPGANGAGNQPGANGAG |
| | GQAAGGNAANKKAGDAGAGQGQNNEGANATNEKSVKEYLDKVR |
| | ATVGTEWTPCSVTCGVGVRVRRRVNAANKKPEDLTLNDLETDVCT |
| | MDKCA |
| 16 | AAGCTTGGGCCCTCGAGCAAGCATATACATAAAAGGGAATATGG |
| Pbcsp 5′HR | |
| forward | |
| 17 | GTCGACTTTAAATATATGCGTGTATATATAGATTTTGTTTTTTG |
| Pbcsp 5′HR | |
| reverse | |
| 18 | CGTCGACATAAACATTACGCATGATTATAAATATTTATA |
| Pbcsp 3′HR | |
| forward | |
| 19 | GAATTCTCGAGAATTAACCAATGCTGTATACTATTATAATGTC |
| Pbcsp 3′HR | |
| reverse | |
| 20 | CCAACACACTGTGGTCACAACG |
| Pvcsp orf | |
| forward | |
| 21 | CAACGTTAAATATACCGGCACACTTATCC |
| Pvcsp orf | |
| reverse | |
| 22 | CAATTCCTCTTTCAATCTGTACATATTTATTCTC |
| Pbcsp | |
| integration | |
| forward | |
| 23 | TCTACGTTGTGACCACAGTGTGTTG |
| Pvcsp | |
| integration | |
| reverse | |
| 24 | CATTGAATGACCTTGAGACTGATGTTTGTAC |
| Pvcsp | |
| integration | |
| forward | |
| 25 | GTATTTTTATTGAAAAAGACACAAAATAGCTAGTTTG |
| Pbcsp | |
| integration | |
| reverse | |
| 26 | GGTATTCCACGCTGTGTTGTCGGTGAG |
| SM forward | |
| 27 | GTTCATAATTTTAGCTATTTACATGCATGTGC |
| SM reverse | |
| 28 | ATGAAGAAGTGTACCATTTTAGTTGTAGCG |
| Pbcsp orf | |
| forward | |
| 29 | GTATCAATATCTTCTAAGGTCAAATCTTCTGC |
| Pbcsp orf | |
| reverse | |
| 30 | TATACACGCATATATTTAAAATGAAAAACTTTATTTTATTAGCAG |
| Pvcsp P01 | TATCTTCTATCTTATTAGTTGACTTATTTCCAACACACTGTGGTCA |
| Sequence | CAACGTAGACTTATCTAAAGCGATAAATTTAAATGGTGTTAACTT |
| | TAATAATGTTGACGCATCATCATTAGGAGCAGCCCACGTTGGTA |
| | AAGCGCATCACGAGGTCGTGGATTAGGTGAAAATCCAGACGACG |
| | AAGAGGGTGACGCAAAAAAAAAAAAGGACGGAAAAAAAGCAG |
| | AACCAAAAAATCCACGTGAAAATAAGTTAAAACAACCAGCAGG |
| | TGACCGAGCAGACGGACAACCAGCAGGTGACCGAGCAGACGGA |
| | CAGCCAGCAGGTGACAGAGCAGACGGACAGCCAGCAGGTGACC |
| | GGGCAGCCGGACAACCAGCAGGTGACAGGGCAGACGGACAGCC |
| | AGCAGGTGACAGGGCAGCGGGACAACCAGCAGGTGACAGAGCA |
| | GACGGACAGCCAGCAGGAGACCGAGCAGCCGGACAACCAGCAG |
| | GTGATCGAGCAGACGGACAGCCAGCAGGAGACAGGGCAGCGGG |
| | ACAACCAGCAGGTGATAGAGCAGCGGGACAACCAGCAGGTGAT |
| | AGAGCAGATGGACAGCCAGCAGGTGATAGGGCAGCTGGACAAC |
| | CAGCAGGTGATAGAGCAGATGGACAACCAGCAGGAGATAGAGC |
| | AGCTGGACAGCCAGCAGGAGATAGAGCAGCTGGACAGCCAGCA |
| | GGAAATGGTGCAGGTGGACAGGCAGCAGGAGGAAACGCAGGAG |
| | GAAACGCAGGAGGAAACGCAGGAGGAAACGCAGGAGGACAGG |
| | GACAAAATAATGAAGGTGCGAATGCCCCAAATGAAAAATCTGTA |
| | AAAGAATACCTAGATAAAGTTAGAGCCACCGTTGGTACCGAATG |
| | GACACCATGTAGTGTAACCTGTGGAGTAGGTGTACGAGTACGAC |
| | GGCGAGTTAATGCAGCGAACAAAAAACCAGAGGATCTTACATTG |
| | AATGACCTTGAGACTGATGTTTGTACAATGGATAAGTGTGCCGG |
| | TATATTTAACGTTGTGAGTAATTCATTAGGTCTAGTCATATTGTT |
| | AGTTCTAGCATTATTCAATTAAATAAACATTACGCATGAT |

### REFERENCES

Price, R. N.; Tjitra, E.; Guerra, C. A.; Yeung, S.; White, N. J.; Anstey, N. M. vivax malaria: neglected and not benign. Am J Trop Med Hyg 2007, 77, 79-87.
Guerra, C. A.; Howes, R. E.; Patil, A. P.; Gething, P. W.; Van Boeckel, T. P.; Temperley, W. H.; Kabaria, C. W.; Tatem, A. J.; Manh, B. H.; Elyazar, I. R.; et al. The international limits and population at risk of Plasmodium vivax transmission in 2009. PLOS neglected tropical diseases 2010, 4, e774, doi: 10.1371/journal.pntd.0000774.
Mendis, K.; Sina, B. J.; Marchesini, P.; Carter, R. The neglected burden of Plasmodium vivax malaria. The American journal of tropical medicine and hygiene 2001, 64, 97-106.
Patarroyo, M. A.; Calderon, D.; Moreno-Perez, D. A. Vaccines against Plasmodium vivax: a research challenge. Expert Rev Vaccines 2012, 11, 1249-1260, doi: 10.1586/erv.12.91.
Barry, A. E.; Arnott, A. Strategies for designing and monitoring malaria vaccines targeting diverse antigens. Front Immunol 2014, 5, 359, doi: 10.3389/fimmu.2014.00359.
Nardin, E. H.; Nussenzweig, V.; Nussenzweig, R. S.; Collins, W. E.; Harinasuta, K. T.; Tapchaisri, P.; Chomcharn, Y. Circumsporozoite proteins of human malaria parasites Plasmodium falciparum and Plasmodium vivax. J Exp Med 1982, 156, 20-30, doi: 10.1084/jem. 156.1.20.
Medica, D. L.; Sinnis, P. Quantitative dynamics of Plasmodium yoelii sporozoite transmission by infected anopheline mosquitoes. Infect Immun 2005, 73, 4363-4369, doi: 10.1128/IAI.73.7.4363-4369.2005. 8. Sinnis, P.; Zavala, F. The skin: where malaria infection and the host immune response begin. Semin Immunopathol 2012, 34, 787-792, doi: 10.1007/s00281-012-0345-5.
Draper, S. J.; Sack, B. K.; King, C. R.; Nielsen, C. M.; Rayner, J. C.; Higgins, M. K.; Long, C. A.; Seder, R. A. Malaria Vaccines: Recent Advances and New Horizons. Cell Host Microbe 2018, 24, 43-56, doi: 10.1016/j.chom.2018.06.008.
Nussenzweig, R. S.; Nussenzweig, V. Development of sporozoite vaccines. Philos Trans R Soc Lond B Biol Sci 1984, 307, 117-128, doi: 10.1098/rstb.1984.0113.
Nussenzweig, R. S.; Vanderberg, J.; Most, H.; Orton, C. Protective immunity produced by the injection of x-irradiated sporozoites of Plasmodium berghei. Nature 1967, 216, 160-162, doi: 10.1038/216160a0.
Nussenzweig, R. S.; Vanderberg, J.; Spitalny, G. L.; Rivera, C. I.; Orton, C.; Most, H. Sporozoiteinduced immunity in mammalian malaria. A review. Am J Trop Med Hyg 1972, 21, 722-728, doi: 10.4269/ajtmh.1972.21.722.
Potocnjak, P.; Yoshida, N.; Nussenzweig, R. S.; Nussenzweig, V. Monovalent fragments (Fab) of monoclonal antibodies to a sporozoite surface antigen (Pb44) protect mice against malarial infection. J Exp Med 1980, 151, 1504-1513, doi: 10.1084/jem. 151.6.1504.
Zavala, F.; Cochrane, A. H.; Nardin, E. H.; Nussenzweig, R. S.; Nussenzweig, V. Circumsporozoite proteins of malaria parasites contain a single immunodominant region with two or more identical epitopes. J Exp Med 1983, 157, 1947-1957, doi: 10.1084/jem. 157.6.1947.
Aly, A. S.; Vaughan, A. M.; Kappe, S. H. Malaria parasite development in the mosquito and infection of the mammalian host. Annu Rev Microbiol 2009, 63, 195-221, i: 10.1146/annurev.micro.091208.073403.
Weiss, W. R.; Berzofsky, J. A.; Houghten, R. A.; Sedegah, M.; Hollindale, M.; Hoffman, S. L. A T cell clone directed at the circumsporozoite protein which protects mice against both Plasmodium yoelii and Plasmodium berghei. J Immunol 1992, 149, 2103-2109.
Frevert, U.; Krzych, U. Plasmodium cellular effector mechanisms and the hepatic microenvironment. Front Microbiol 2015, 6, 482, doi: 10.3389/fmich.2015.00482.
Roth, A.; Maher, S. P.; Conway, A. J.; Ubalee, R.; Chaumeau, V.; Andolina, C.; Kaba, S. A.; Vantaux, A.; Bakowski, M. A.; Thomson-Luque, R.; et al. A comprehensive model for assessment of liver stage therapies targeting Plasmodium vivax and Plasmodium falciparum. Nat Commun 2018, 9, 1837, doi: 10.1038/s41467-018-04221-9.
RTS, S. C. T. P. Efficacy and safety of RTS,S/AS01 malaria vaccine with or without a booster dose in infants and children in Africa: final results of a phase 3, individually randomised, controlled trial. Lancet 2015, 386, 31-45, doi: 10.1016/s0140-6736 (15) 60721-8.
White, M. T.; Verity, R.; Griffin, J. T.; Asante, K. P.; Owusu-Agyei, S.; Greenwood, B.; Drakeley, C.; Gesase, S.; Lusingu, J.; Ansong, D.; et al. Immunogenicity of the RTS,S/AS01 malaria vaccine and implications for duration of vaccine efficacy: secondary analysis of data from a phase 3 randomised controlled trial. The Lancet Infectious Diseases 2015, 15, 1450-1458, doi: 10.1016/S1473-3099 (15) 00239-X.
Chandramohan, D.; Zongo, I.; Sagara, I.; Cairns, M.; Yerbanga, R.; Diarra, M.; Nikiema, F.; Tapily, A.; Sompougdou, F.; Issiaka, D.; et al. Seasonal Malaria Vaccination with or without Seasonal Malaria Chemoprevention. N. Engl. J. Med. 2021, 385, 1005-1017, doi: 10.1056/NEJMoa2026330.
Bennett, J. W.; Yadava, A.; Tosh, D.; Sattabongkot, J.; Komisar, J.; Ware, L. A.; McCarthy, W. F.; Cowden, J. J.; Regules, J.; Spring, M. D.; et al. Phase 1/2a Trial of Plasmodium vivax Malaria Vaccine Candidate VMP001/AS01B in Malaria-Naive Adults: Safety, Immunogenicity, and Efficacy. PLOS Negl. Trop. Dis. 2016, 10, e0004423, doi: 10.1371/journal.pntd. 0004423.
Atcheson, E.; Bauza, K.; Salman, A. M.; Alves, E.; Blight, J.; Viveros-Sandoval, M. E.; Janse, C. J.; Khan, S. M.; Hill, A. V. S.; Reyes-Sandoval, A. Tailoring a Plasmodium vivax Vaccine To Enhance Efficacy through a Combination of a CSP Virus-Like Particle and TRAP Viral Vectors. Infect. Immun. 2018, 86, e00114-00118, doi: 10.1128/IAI.00114-18.
Herrington, D. A.; Nardin, E. H.; Losonsky, G.; Bathurst, I. C.; Barr, P. J.; Hollingdale, M. R.; Edelman, R.; Levine, M. M. Safety and Immunogenicity of a Recombinant Sporozoite Malaria Vaccine against Plasmodium vivax. Am J Trop Med Hyg 1991, 45, 695-701, doi: 10.4269/ajtmh. 1991.45.695.
Herrera, S.; De Plata, C.; González, M.; Perlaza, B. L.; Bettens, F.; Corradin, G.; Arévalo-Herrera, M. Antigenicity and immunogenicity of multiple antigen peptides (MAP) containing P. vivax CS epitopes in Aotus monkeys. Parasite Immunol 1997, 19, 161-170, doi: 10.1046/j. 1365-3024.1997.d01-193.x.
Herrera, S.; Bonelo, A.; Liliana Perlaza, B.; Zully Valencia, A.; Cifuentes, C.; Hurtado, S.; Quintero, G.; Alejandro López, J.; Corradin, G.; Arévalo-Herrera, M. Use of long synthetic peptides to study the antigenicity and immunogenicity of the Plasmodium vivax circumsporozoite protein. International Journal for Parasitology 2004, 34, 1535-1546, doi: 10.1016/j.ijpara.2004.10.009.
Herrera, S.; Bonelo, A.; Perlaza, B. L.; Fernández, O. L.; Victoria, L.; Lenis, A. M.; Soto, L.; Hurtado, H.; Acuña, L. M.; Vélez, J. D.; et al. Safety and elicitation of humoral and cellular responses in Colombian malaria-naive volunteers by a Plasmodium vivax circumsporozoite protein-derived synthetic vaccine. Am J Trop Med Hyg 2005, 73, 3-9, i: 10.4269/ajtmh.2005.73.3.
Herrera, S.; Fernandez, O. L.; Vera, O.; Cardenas, W.; Ramirez, O.; Palacios, R.; Chen-Mok, M.; Corradin, G.; Arevalo-Herrera, M. Phase I safety and immunogenicity trial of Plasmodium vivax CS derived long synthetic peptides adjuvanted with montanide ISA 720 or montanide ISA 51. Am J Trop Med Hyg 2011, 84, 12-20, doi: 10.4269/ajtmh.2011.09-0516.
Atcheson, E.; Reyes-Sandoval, A. Protective efficacy of peptides from Plasmodium vivax circumsporozoite protein. Vaccine 2020, 38, 4346-4354, doi: 10.1016/j.vaccine.2020.03.063. 30. Chatterjee, D.; Cockburn, I. A. The challenges of a circumsporozoite protein-based malaria vaccine. Expert Rev Vaccines 2021, 20, 113-125, doi: 10.1080/14760584.2021.1874924.
De, S. L.; Ntumngia, F. B.; Nicholas, J.; Adams, J. H. Progress towards the development of a P. vivax vaccine. Expert Rev. Vaccines 2021, 20, 97-112, doi: 10.1080/14760584.2021.1880898. 32. Braga, E. M.; Fontes, C. J.; Krettli, A. U. Persistence of humoral response against sporozoite and blood-stage malaria antigens 7 years after a brief exposure to Plasmodium vivax. J Infect Dis 1998, 177, 1132-1135, doi: 10.1086/517412.
Wongsrichanalai, C.; Webster, H. K.; Permpanich, B.; Chuanak, N.; Ketrangsri, S. Naturally acquired circumsporozoite antibodies and their role in protection in endemic falciparum and vivax malaria. Am J Trop Med Hyg 1991, 44, 201-204, doi: 10.4269/ajtmh. 1991.44.201. 34. Longley, R. J.; Reyes-Sandoval, A.; Montoya-Diaz, E.; Dunachie, S.; Kumpitak, C.; Nguitragool, W.; Mueller, I.; Sattabongkot, J. Acquisition and Longevity of Antibodies to Plasmodium vivax Preerythrocytic Antigens in Western Thailand. Clin Vaccine Immunol 2016, 23, 117-124, doi: 10.1128/CVI.00501-15.
Soares, I. F.; Lopez-Camacho, C.; Rodrigues-da-Silva, R. N.; da Silva Matos, A.; de Oliveira Baptista, B.; Totino, P. R. R.; de Souza, R. M.; Harrison, K.; Gimenez, A. M.; de Freitas, E. O.; et al. Recombinant Plasmodium vivax circumsporozoite surface protein allelic variants: antibody recognition by individuals from three communities in the Brazilian Amazon. Sci Rep 2020, 10, 14020, doi: 10.1038/s41598-020-70893-3.
Ntumngia, F. B.; Kolli, S. K.; Annamalai Subramani, P.; Barnes, S. J.; Nicholas, J.; Ogbondah, M. M.; Barnes, B. B.; Salinas, N. D.; Thawornpan, P.; Tolia, N. H.; et al. Naturally acquired antibodies against Plasmodium vivax pre-erythrocytic stage vaccine antigens inhibit sporozoite invasion of human hepatocytes in vitro. Sci Rep 2024, 14, 1260, doi: 10.1038/s41598-024-51820-2.
Kheirollahpour, M.; Mehrabi, M.; Dounighi, N. M.; Mohammadi, M.; Masoudi, A. Nanoparticles and Vaccine Development. Pharm Nanotechnol 2020, 8, 6-21, doi: 10.2174/2211738507666191024162042.
Tukhvatulin, A.; Dzharullaeva, A.; Erokhova, A.; Zemskaya, A.; Balyasin, M.; Ozharovskaia, T.; Zubkova, O.; Shevlyagina, N.; Zhukhovitsky, V.; Fedyakina, I.; et al. Adjuvantation of an Influenza Hemagglutinin Antigen with TLR4 and NOD2 Agonists Encapsulated in Poly(D,L-Lactide-Co-Glycolide) Nanoparticles Enhances Immunogenicity and Protection against Lethal Influenza Virus Infection in Mice. Vaccines (Basel) 2020, 8, doi: 10.3390/vaccines8030519.
Quach, Q. H.; Ang, S. K.; Chu, J. J.; Kah, J. C. Y. Size-dependent neutralizing activity of gold nanoparticle-based subunit vaccine against dengue virus. Acta Biomater 2018, 78, 224-235, doi: 10.1016/j.actbio.2018.08.011. 40. Cohen, K. W.; De Rosa, S. C.; Fulp, W. J.; deCamp, A. C.; Fiore-Gartland, A.; Mahoney, C. R.; Furth, S.; Donahue, J.; Whaley, R. E.; Ballweber-Fleming, L.; et al. A first-in-human germline-targeting HIV nanoparticle vaccine induced broad and publicly targeted helper T cell responses. Sci Transl Med 2023, 15, eadf3309, doi: 10.1126/scitranslmed.adf3309.
Angioletti-Uberti, S. Exploiting Receptor Competition to Enhance Nanoparticle Binding Selectivity. Phys Rev Lett 2017, 118, 068001, doi: 10.1103/PhysRevLett. 118.068001.
Zhao, L.; Seth, A.; Wibowo, N.; Zhao, C. X.; Mitter, N.; Yu, C.; Middelberg, A. P. Nanoparticle vaccines. Vaccine 2014, 32, 327-337, doi: 10.1016/j.vaccine.2013.11.069.
Chavda, V. P.; Yao, Q.; Vora, L. K.; Apostolopoulos, V.; Patel, C. A.; Bezbaruah, R.; Patel, A. B.; Chen, Z. S. Fast-track development of vaccines for SARS-COV-2: The shots that saved the world. Front Immunol 2022, 13, 961198, doi: 10.3389/fimmu.2022.961198.
De Temmerman, M. L.; Rejman, J.; Demeester, J.; Irvine, D. J.; Gander, B.; De Smedt, S. C. Particulate vaccines: on the quest for optimal delivery and immune response. Drug Discov Today 2011, 16, 569-582, doi: 10.1016/j.drudis.2011.04.006.
Butkovich, N.; Li, E.; Ramirez, A.; Burkhardt, A. M.; Wang, S. W. Advancements in protein nanoparticle vaccine platforms to combat infectious disease. Wiley Interdiscip Rev Nanomed Nanobiotechnol 2021, 13, e1681, doi: 10.1002/wnan.1681.
Bachmann, M. F.; Jennings, G. T. Vaccine delivery: a matter of size, geometry, kinetics and molecular patterns. Nat Rev Immunol 2010, 10, 787-796, doi: 10.1038/nri2868.
Machhi, J.; Shahjin, F.; Das, S.; Patel, M.; Abdelmoaty, M. M.; Cohen, J. D.; Singh, P. A.; Baldi, A.; Bajwa, N.; Kumar, R.; et al. Nanocarrier vaccines for SARS-COV-2. Adv Drug Deliv Rev 2021, 171, 215-239, doi: 10.1016/j.addr.2021.01.002.
Chattopadhyay, S.; Chen, J. Y.; Chen, H. W.; Hu, C. J. Nanoparticle Vaccines Adopting Virus-like Features for Enhanced Immune Potentiation. Nanotheranostics 2017, 1, 244-260, doi: 10.7150/ntno. 19796.
Gregory, A. E.; Titball, R.; Williamson, D. Vaccine delivery using nanoparticles. Front Cell Infect Microbiol 2013, 3, 13, doi: 10.3389/fcimb.2013.00013.
Moon, J. J.; Suh, H.; Polhemus, M. E.; Ockenhouse, C. F.; Yadava, A.; Irvine, D. J. Antigen-displaying lipid-enveloped PLGA nanoparticles as delivery agents for a Plasmodium vivax malaria vaccine. PLOS One 2012, 7, e31472, doi: 10.1371/journal.pone.0031472.
Thomas, C.; Rawat, A.; Hope-Weeks, L.; Ahsan, F. Aerosolized PLA and PLGA nanoparticles enhance humoral, mucosal and cytokine responses to hepatitis B vaccine. Mol Pharm 2011, 8, 405-415, doi: 10.1021/mp100255c.
Manish, M.; Rahi, A.; Kaur, M.; Bhatnagar, R.; Singh, S. A single-dose PLGA encapsulated protective antigen domain 4 nanoformulation protects mice against Bacillus anthracis spore challenge. PLOS One 2013, 8, e61885, doi: 10.1371/journal.pone.0061885.
Kucharska, I.; Hossain, L.; Ivanochko, D.; Yang, Q.; Rubinstein, J. L.; Pomes, R.; Julien, J. P. Structural basis of Plasmodium vivax inhibition by antibodies binding to the circumsporozoite protein repeats. Elife 2022, 11, doi: 10.7554/eLife.72908.
Vo, T. C.; Trinh, N. T. M.; Le, H. G.; Kang, J. M.; Yoo, W. G.; Quang, H. H.; Na, B. K. Genetic Diversity of Circumsporozoite Surface Protein of Plasmodium vivax from the Central Highlands, Vietnam. Pathogens 2022, 11, doi: 10.3390/pathogens11101158.
Mizutani, M.; Fukumoto, S.; Soubeiga, A. P.; Soga, A.; Iyori, M.; Yoshida, S. Development of a Plasmodium berghei transgenic parasite expressing the full-length Plasmodium vivax circumsporozoite VK247 protein for testing vaccine efficacy in a murine model. Malar J 2016, 15,251, doi: 10.1186/s12936-016-1297-3.
Rosenberg, R.; Wirtz, R. A.; Lanar, D. E.; Sattabongkot, J.; Hall, T.; Waters, A. P.; Prasittisuk, C. Circumsporozoite protein heterogeneity in the human malaria parasite Plasmodium vivax. Science 1989, 245, 973-976, doi: 10.1126/science.2672336.
Gonzalez, J. M.; Hurtado, S.; Arevalo-Herrera, M.; Herrera, S. Variants of the Plasmodium vivax circumsporozoite protein (VK210 and VK247) in Colombian isolates. Mem Inst Oswaldo Cruz 2001, 96, 709-712, doi: 10.1590/s0074-02762001000500023.
Arnot, D. E.; Barnwell, J. W.; Tam, J. P.; Nussenzweig, V.; Nussenzweig, R. S.; Enea, V. Circumsporozoite protein of Plasmodium vivax: gene cloning and characterization of the immunodominant epitope. Science 1985, 230, 815-818, doi: 10.1126/science.2414847.
Gonzalez-Ceron, L.; Rodriguez, M. H.; Wirtz, R. A.; Sina, B. J.; Palomeque, O. L.; Nettel, J. A.; Tsutsumi, V. Plasmodium vivax: a monoclonal antibody recognizes a circumsporozoite protein precursor on the sporozoite surface. Exp Parasitol 1998, 90, 203-211, doi: 10.1006/expr. 1998.4334.

What is claimed is:

1. An engineered recombinant P. vivax circumsporozoite surface protein (CSP) of strain VK210 or VK247, or a fragment thereof.

2. The engineered recombinant P. vivax CSP of claim 1, wherein the CSP comprises a central repeat region (CRR) with the amino acid sequence as set forth in SEQ ID NO: 1 or SEQ ID NO: 2, or functional homologues thereof, having at least 90% identity with SEQ ID NO: 1 or SEQ ID NO: 2.

3. The engineered recombinant P. vivax CSP of claim 1, wherein the CSP comprises a central repeat region (CRR) with the amino acid sequence as set forth in SEQ ID NO: 4 or SEQ ID NO: 5, or functional homologues thereof, having at least 90% identity with SEQ ID NO: 4 or SEQ ID NO: 5.

4. The engineered recombinant P. vivax CSP of claim 1, wherein the CSP comprises a full length CSP with the amino acid sequence as set forth in SEQ ID NO: 7 or SEQ ID NO: 8, or functional homologues thereof, having at least 90% identity with SEQ ID NO: 7 or SEQ ID NO: 8.

5. The engineered recombinant P. vivax CSP of claim 1, wherein the CSP comprises the N-terminal amino acid sequence as set forth in SEQ ID NO: 10, or functional homologues thereof, having at least 90% identity with SEQ ID NO: 10.

6. The engineered recombinant P. vivax CSP of claim 1, wherein the CSP comprises the C-terminal amino acid sequence as set forth in SEQ ID NO: 11, or functional homologues thereof, having at least 90% identity with SEQ ID NO: 11.

7. The engineered recombinant P. vivax CSP of claim 1, wherein the CSP comprises the N-terminal and C-terminal amino acid sequence as set forth in SEQ ID NO: 12, or functional homologues thereof, having at least 90% identity with SEQ ID NO: 12.

8. A nucleic acid encoding the engineered CSPs of claim 2.

9. A nucleic acid encoding the engineered CSPs of claim 3.

10. A nucleic acid encoding the engineered CSPs of claim 4.

11. A nucleic acid encoding the engineered CSPs of claim 5.

12. A nucleic acid encoding the engineered CSPs of claim 6.

13. A nucleic acid encoding the engineered CSPs of claim 7.

14. A nanoparticle vaccine comprising the engineered CSP of claim 2 and a pharmaceutically acceptable carrier.

15. A nanoparticle vaccine comprising the engineered CSP of claim 3 and a pharmaceutically acceptable carrier.

16. A nanoparticle vaccine comprising the engineered CSP of claim 4 and a pharmaceutically acceptable carrier.

17. A nanoparticle vaccine comprising the engineered CSP of claim 5 and a pharmaceutically acceptable carrier.

18. A nanoparticle vaccine comprising the engineered CSP of claim 6 and a pharmaceutically acceptable carrier.

19. A nanoparticle vaccine comprising the engineered CSP of claim 7 and a pharmaceutically acceptable carrier.

20. A method of treating or preventing malaria disease in a subject in need thereof, the method comprising administering a therapeutically or prophylactically effective amount of the engineered CSP, or a fragment thereof, of claim 1 to the subject.
